(19) United States
(12) Patent Application Publication
(10) Pub. No.: US 2025/0250214 A1
(43) Pub. Date: Aug. 7, 2025

(54) METHOD FOR DEHYDRATION OF ALCOHOL MIXTURES

(71) Applicant: Braskem S.A., Sao Paulo (BR)

(72) Inventors: Andrea MARINS de OLIVEIRA, Sao Paulo (BR); Robson Pablo SOBRADIEL PEGUIN, Sao Paulo (BR); Nailma de JESUS MARTINS, Sao Paulo (BR); Jieun LEE, Sao Paulo (BR)

(21) Appl. No.: 19/047,156

(22) Filed: Feb. 6, 2025

Related U.S. Application Data
(60) Provisional application No. 63/550,958, filed on Feb. 7, 2024.

(51) Int. Cl. C07C 1/24; B01D 3/14; B01J 21/04; B01J 35/61; B01J 35/63; C07C 7/04

(52) U.S. Cl. CPC C07C 1/24; B01D 3/143; B01J 21/04; B01J 35/613; B01J 35/615; B01J 35/635; B01J 35/638; C07C 7/04; C07C 2521/04

(57) ABSTRACT

The present disclosure provides processes for production of ethylene and propylene. The processes comprise contacting a mixture of ethanol and at least one C₃ alcohol with a dehydration catalyst in a dehydration unit, withdrawing from said dehydration unit a stream containing ethylene and propylene, and separating the ethylene and the propylene from the stream, wherein the mixture of ethanol and at least one C₃ alcohol has a water content of 0 to 75% by weight of the total weight of the mixture, wherein the mixture is produced by fermentation using a recombinant yeast in one or more fermenters, and wherein the mixture contains at least 50% ethanol by weight of the total weight of ethanol and C₃ alcohols.

### CROSS-REFERENCE TO RELATED APPLICATIONS

This application claims priority to U.S. Provisional Application No. 63/550,958, filed Feb. 7, 2024, which is incorporated herein by reference in its entirety.

### BACKGROUND

Olefins are important intermediates for the chemical industry. Traditionally, olefins have been obtained by steam cracking of hydrocarbons. However, increased crude oil prices and instability in crude oil supply sources have made traditional steam cracking processes less cost competitive compared to alternative approaches for producing olefins.

One such approach is the dehydration of alcohols to obtain olefins. Advantageously, the raw materials for alcohol dehydration can be obtained from renewable and sustainable sources such as by fermentation of diversified bio-resources including sugar cane, corn, agricultural and cellulosic biomass, and algae-based feedstocks.

U.S. Pat. No. 9,902,662 discloses a method for dehydrating a mixture containing ethanol and n-propanol.

U.S. Pat. No. 9,902,663 discloses a method for dehydrating a mixture containing ethanol and isopropanol.

Nonetheless, there exists a need for alternative dehydration processes, including green processes that produce ethylene and propylene, from alcohol mixtures obtained from biological processes. Further, there exists a need for alternative dehydration processes that are higher yielding, more efficient, and require fewer purification and/or processing steps. Further still, there exists a need to flexibilize future alcohol dehydration assets to produce olefins on demand.

### SUMMARY OF THE DISCLOSURE

The present disclosure provides processes for production of ethylene and propylene. In an embodiment, the disclosed processes are achieved by designing mixed alcohol dehydration units and changing the alcohol composition in function of production of desirable olefin(s).

In one aspect, the disclosure provides a process for production of ethylene and propylene, the process comprising: (a) contacting a mixture of ethanol and at least one C₃ alcohol with a dehydration catalyst in a dehydration unit at a temperature of 250° C. to 500° C. and at a total pressure of 2 bar to 20 bar to produce ethylene and propylene; (b) withdrawing from said dehydration unit a stream containing ethylene and propylene produced from step (a); and (c) separating the ethylene and the propylene from the stream obtained from step (b); wherein the mixture of ethanol and at least one C₃ alcohol has a water content of 0 to 75% by weight of the total weight of the mixture, wherein the mixture is produced by fermentation using a recombinant yeast in one or more fermenters, and wherein the mixture contains at least 50% ethanol by weight of the total weight of ethanol and C₃ alcohols. In an embodiment, the mixture of ethanol and at least one C₃ alcohol has a water content of 0 to 25% by weight of the total weight of the mixture. In an embodiment, the contacting in the dehydration unit is at a temperature of 250° C. to 480° C., such as 250° C. to 300° C. In an embodiment, the mixture comprises 1-propanol and/or isopropanol.

In an embodiment, the ethanol and at least one C₃ alcohol are produced from a fermentable carbon source.

In an embodiment, the recombinant yeast is an ethanol-producing yeast. In an embodiment, the ethanol-producing yeast is a genetically modified Saccharomyces cerevisiae.

In an embodiment, the recombinant yeast produces the at least one C₃ alcohol by expression of one or more exogenous genes.

In an embodiment, the recombinant ethanol-producing yeast comprises: (i) one or more polynucleotides coding for enzymes in a pathway that catalyze a conversion of the fermentable carbon source to succinyl-CoA; (ii) one or more polynucleotides coding for enzymes in a pathway that catalyze a conversion of succinyl-CoA to methylmalonyl-CoA; (iii) one or more polynucleotides coding for enzymes in a pathway that catalyze a conversion of methylmalonyl-CoA to propionyl-CoA; (iv) one or more polynucleotides coding for enzymes in a pathway that catalyze a conversion of propionyl-CoA to propionaldehyde; and (v) one or more polynucleotides coding for enzymes in a pathway that catalyze a conversion of propionaldehyde to 1-propanol.

In an embodiment, the recombinant ethanol-producing yeast comprises: (i) one or more polynucleotides coding for enzymes in a pathway that catalyze a conversion of the fermentable carbon source to 1,2-propanediol; (ii) one or more polynucleotides coding for enzymes in a pathway that catalyze a conversion of 1,2-propanediol to propionaldehyde; and (iii) one or more polynucleotides coding for enzymes in a pathway that catalyze a conversion of propionaldehyde to 1-propanol.

In an embodiment, the recombinant ethanol-producing yeast comprises: (i) one or more polynucleotides coding for enzymes in a pathway that catalyze a conversion of the fermentable carbon source to pyruvate or malonate semialdehyde (MSA); (ii) one or more polynucleotides coding for enzymes in a pathway that catalyze a conversion of pyruvate or MSA to acetyl-CoA; (iii) one or more polynucleotides coding for enzymes in a pathway that catalyze a conversion of acetyl-CoA to acetoacetyl-CoA; (iv) one or more polynucleotides coding for enzymes in a pathway that catalyze a conversion of acetoacetyl-CoA to acetoacetate; and (v) one or more polynucleotides coding for enzymes in a pathway that catalyze a conversion of acetoacetate to acetone; and (vi) one or more polynucleotides coding for enzymes in a pathway that catalyze a conversion of acetone to isopropanol (2-propanol).

In an embodiment, the recombinant ethanol-producing yeast comprises: (i) one or more polynucleotides coding for enzymes in a pathway that catalyze a conversion of the fermentable carbon source to a phosphate intermediate; (ii) one or more polynucleotides coding for enzymes in a pathway that catalyze the conversion of D-xylulose 5-phosphate to D-glyceraldehyde 3-phosphate and acetyl phosphate, or the conversion of D-fructose 6-phosphate to D-erythrose 4-phosphate and acetyl phosphate; (iii) one or more polynucleotides coding for enzymes in a pathway that catalyze the conversion of acetyl phosphate to acetyl-CoA; or one or more polynucleotides coding for enzymes in a pathway that catalyze the conversion of acetyl phosphate to acetate and one or more polynucleotides coding for enzymes in a pathway that catalyze the conversion of acetate to acetyl-CoA; (iv) one or more polynucleotides coding for enzymes in a pathway that catalyze a conversion of acetyl-CoA to acetoacetyl-CoA; (v) one or more polynucleotides coding for enzymes in a pathway that catalyze a conversion of acetoacetyl-CoA to acetoacetate; (vi) one or more polynucleotides coding for enzymes in a pathway that catalyze a conversion of acetoacetate to acetone; and (vii) one or more polynucleotides coding for enzymes in a pathway that catalyze a conversion of acetone to isopropanol (2-propanol).

In an embodiment, the mixture of ethanol and at least one C₃ alcohol has a water content of 0 and 25% by weight of the total weight of the mixture.

In an embodiment, the mixture contains at least 70% ethanol by weight of the total weight of ethanol and C₃ alcohols.

In an embodiment, the contacting in the dehydration unit is at a temperature of 350° C. to 480° C. and at a total pressure of 5 bar to 15 bar.

In an embodiment, the dehydration catalyst is selected from zeolites, oxides, heteropolyacids, or a mixture thereof. In an embodiment, the zeolite catalyst is selected from ZSM-5, MOR, or FER. In an embodiment, the heteropolyacid catalyst is selected from Ag₃PW₁₂O₄₀, K₂HPW₁₂O₄₀, or Cs₃PMo₁₂O₄₀. In an embodiment, the oxide catalyst is selected from gamma alumina, eta alumina, or chi alumina. In an embodiment, the gamma alumina has a surface area of 90 m²/g to 200 m²/g, pore volume of 0.45 to 0.70 cm³/g and average pore size width of 9 Angstroms to 11 Angstroms. In an embodiment, the gamma alumina has a surface area of 90 m²/g to 170 m²/g, pore volume of 0.45 to 0.55 cm³/g and average pore size width of 9 Angstroms to 11 Angstroms. In an embodiment, the gamma alumina has a surface area of 350 m²/g to 450 m²/g, pore volume of 1.00 to 1.20 cm³/g and average pore size width of 20 Angstroms to 30 Angstroms.

In an embodiment, the dehydration unit consists of two or more reactors which are positioned in series and/or in parallel. In an embodiment, the reactors are adiabatic and/or isothermal.

In an embodiment, the mixture further comprises one or more of acetone, salts, heavy components, solids and other contaminants.

In an embodiment, the mixture is treated by a separation system before contacting the mixture in step (a). In an embodiment, the mixture is treated by a distillation system before contacting the mixture in step (a).

In an embodiment, the separation system comprises at least first and second separation units. In an embodiment, the separation system comprises 2 or more separation units, such as 2 to 10 separation units, 2 to 9 separation units, 2 to 8 separation units, 2 to 7 separation units, 2 to 6 separation units, 2 to 5 separation units, 2 to 4 separation units, 3 separation units, 4 separation units, 5 separation units, 6 separation units, 7 separation units, 8 separation units, 9 separation units, or 10 separation units. In an embodiment, the first separation unit is an evaporator, a distillation column, a set of distillation columns, a combination of a centrifuge and a distillation column, or a combination of a centrifuge and a set of distillation columns. In an embodiment, the second separation unit is a rectifier column, a distillation column, or a set of distillation columns. In an embodiment, the process further comprises flowing a fermentation off-gas coming from the one or more fermenters through a product recovery unit wherein the flow of the off-gas is counter to a solvent flowing through the product recovery unit and obtaining a solvent stream comprising the at least one C₃ alcohol and ethanol, and mixing the solvent stream with a fermentation broth from the one or more fermenters. In an embodiment, the process further comprises passing the fermentation broth through a first distillation column to generate a bottom stream comprising water, heavy components, and solids, and a side stream comprising ethanol, the at least one C₃ alcohol, and water.

In an embodiment, the side stream from the first distillation column is passed through a second distillation column to generate a bottom stream comprising water and a side stream comprising ethanol, the at least one C₃ alcohol, and water. In an embodiment, the side stream from the second distillation column is passed through a third distillation column if acetone is present at a concentration higher than 2% by weight of the total weight of the mixture, wherein the acetone is recovered at the top of the third distillation column and ethanol, the at least one C₃ alcohol, and water are recovered at the bottom of the distillation column.

In an embodiment, water is further removed by a drying/dewatering unit after the separation system and before contacting the mixture in step (a). In an embodiment, the drying/dewatering unit comprises a membrane separation system and/or a molecular sieve system. In an embodiment, the mixture is treated with an ionic resin system to remove salts after the drying/dewatering unit and before contacting the mixture in step (a). In an embodiment, the ionic resin system comprises a cationic resin, an anionic resin, or a combination thereof.

In an embodiment, the solvent is water.

In an embodiment, water is further added up to 75% in the dehydration unit.

In an embodiment, water is further added up to 25% in the dehydration unit.

In an embodiment, there is no addition of water in the dehydration unit.

In an embodiment, the ethylene and the propylene are separated by distillation, filtration, liquid-liquid separation, or a combination thereof.

In an embodiment, the ethylene and the propylene are polymerized to make polyethylene, polypropylene, and/or a co-polymer of ethylene and propylene.

In an embodiment, the ethylene and the propylene are not separated and are polymerized to make a co-polymer of ethylene and propylene.

In one aspect the disclosure provides a process for production of ethylene and propylene, the process comprising: (a) contacting a mixture of ethanol and isopropanol with a dehydration catalyst in a dehydration unit at a temperature of 250° C. to 500° C. and a total pressure of 2 bar to 20 bar to produce ethylene and propylene; (b) withdrawing from said dehydration unit a stream containing ethylene and propylene produced from step (a); and (c) separating the ethylene and the propylene from the stream obtained from step (b); wherein the mixture of ethanol and isopropanol has a water content of 0 to 75% by weight of the total weight of the mixture, wherein the mixture is produced by fermentation using a recombinant yeast in one or more fermenters, and wherein the mixture contains at least 70% ethanol by weight of the total weight of ethanol and isopropanol.

In one aspect the disclosure provides a process for the production of ethylene and propylene, the process comprising: (a) contacting a mixture of ethanol and 1-propanol with a dehydration catalyst in a dehydration unit at a temperature of 250° C. to 500° C. and a total pressure of 2 bar to 20 bar to produce ethylene and propylene; (b) withdrawing from said dehydration unit a stream containing ethylene and propylene produced from step (a); and (c) separating the ethylene and the propylene from the stream obtained from step (b); wherein the mixture of ethanol and 1-propanol has a water content of 0 to 75% by weight of the total weight of the mixture, wherein the mixture is produced by fermentation using a recombinant yeast in one or more fermenters, and wherein the mixture contains at least 70% ethanol by weight of the total weight of ethanol and 1-propanol.

In one aspect the disclosure provides a process for the production of ethylene and propylene, the process comprising: (a) contacting a mixture of ethanol, isopropanol and 1-propanol with a dehydration catalyst in a dehydration unit at a temperature of 250° C. to 500° C. and a total pressure of 2 bar to 20 bar to produce ethylene and propylene; (b) withdrawing from said dehydration unit a stream containing ethylene and propylene produced from step (a); and (c) separating the ethylene and the propylene from the stream obtained from step (b); wherein the mixture of ethanol, isopropanol and 1-propanol has a water content of 0 to 75% by weight of the total weight of the mixture, wherein the mixture is produced by fermentation using a recombinant yeast in one or more fermenters, and wherein the mixture contains at least 70% ethanol by weight of the total weight of ethanol, isopropanol and 1-propanol.

### BRIEF DESCRIPTION OF THE FIGURES

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 is a process flowsheet representation for the first part of the process in which fermentation products are recovered.

FIG. 2 is a process flowsheet representation for the second part of the process in which water content is reduced from high to low water content.

FIG. 3 is a graph showing the formation of ethylene and propylene over time.

### DETAILED DESCRIPTION OF THE DISCLOSURE

As set forth herein, the disclosure provides methods for the dehydration of alcohols such as ethanol and at least one C₃ alcohol.

### Definitions

As used herein, the term “derived from” may encompass the terms originated from, obtained from, obtainable from, isolated from, and created from, and generally indicates that one specified material finds its origin in another specified material or has features that can be described with reference to another specified material.

As used herein, “exogenous polynucleotide” refers to any deoxyribonucleic acid that originates outside of the microorganism.

As used herein, the term “an expression vector” may refer to a DNA construct containing a polynucleotide or nucleic acid sequence encoding a polypeptide or protein, such as a DNA coding sequence (e.g. gene sequence) that is operably linked to one or more suitable control sequence(s) capable of affecting expression of the coding sequence in a host. Such control sequences include a promoter to affect transcription, an optional operator sequence to control such transcription, a sequence encoding suitable mRNA ribosome binding sites, and sequences which control termination of transcription and translation. The vector may be a plasmid, cosmid, phage particle, bacterial artificial chromosome, or simply a potential genomic insert. Once transformed into a suitable host, the vector may replicate and function independently of the host genome (e.g., independent vector or plasmid), or may, in some instances, integrate into the genome itself (e.g., integrated vector). The plasmid is the most commonly used form of expression vector. However, the disclosure is intended to include such other forms of expression vectors that serve equivalent functions and which are, or become, known in the art.

As used herein, the term “expression” may refer to the process by which a polypeptide is produced based on a nucleic acid sequence encoding the polypeptides (e.g., a gene). The process includes both transcription and translation.

As used herein, the term “gene” may refer to a DNA segment that is involved in producing a polypeptide or protein (e.g., fusion protein) and includes regions preceding and following the coding regions as well as intervening sequences (introns) between individual coding segments (exons).

As used herein, the term “heterologous,” with reference to a nucleic acid, polynucleotide, protein or peptide, may refer to a nucleic acid, polynucleotide, protein or peptide that does not naturally occur in a specified cell, e.g., a host cell. It is intended that the term encompass proteins that are encoded by naturally occurring genes, mutated genes, and/or synthetic genes. In contrast, the term homologous, with reference to a nucleic acid, polynucleotide, protein or peptide, refers to a nucleic acid, polynucleotide, protein or peptide that occurs naturally in the cell.

As used herein, the term a “host cell” may refer to a cell or cell line, including a cell such as a microorganism which a recombinant expression vector may be transfected for expression of a polypeptide or protein (e.g., fusion protein). Host cells include progeny of a single host cell, and the progeny may not necessarily be completely identical (in morphology or in total genomic DNA complement) to the original parent cell due to natural, accidental, or deliberate mutation. A host cell may include cells transfected or transformed in vivo with an expression vector.

As used herein, the term “introduced,” in the context of inserting a nucleic acid sequence or a polynucleotide sequence into a cell, may include transfection, transformation, or transduction and refers to the incorporation of a nucleic acid sequence or polynucleotide sequence into a eukaryotic or prokaryotic cell wherein the nucleic acid sequence or polynucleotide sequence may be incorporated into the genome of the cell (e.g., chromosome, plasmid, plastid, or mitochondrial DNA), converted into an autonomous replicon, or transiently expressed.

As used herein, the term “non-naturally occurring” or “modified” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Non-naturally occurring microbial organisms of the disclosure can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely. Those skilled in the art will understand that the genetic alterations, including metabolic modifications exemplified herein, are described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. Such genetic alterations include, for example, genetic alterations of species homologs, in general, and in particular, orthologs, paralogs or nonorthologous gene displacements.

As used herein, the term “operably linked” may refer to a juxtaposition or arrangement of specified elements that allows them to perform in concert to bring about an effect. For example, a promoter may be operably linked to a coding sequence if it controls the transcription of the coding sequence.

As used herein, “1-propanol” is intended to mean n-propanol with a general formula CH₃CH₂CH₂OH (CAS number-71-23-8).

As used herein, “2-propanol” is intended to mean isopropyl alcohol with a general formula CH₃CH₃CHOH (CAS number-67-63-0).

As used herein, the term “a promoter” may refer to a regulatory sequence that is involved in binding RNA polymerase to initiate transcription of a gene. A promoter may be an inducible promoter or a constitutive promoter. An inducible promoter is a promoter that is active under environmental or developmental regulatory conditions.

As used herein, the term “a polynucleotide” or “nucleic acid sequence” may refer to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Such polynucleotides or nucleic acid sequences may encode amino acids (e.g., polypeptides or proteins such as fusion proteins). Because the genetic code is degenerate, more than one codon may be used to encode a particular amino acid, and the present disclosure encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2'-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms polynucleotide, nucleic acid, and oligonucleotide are used herein interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (thioate), P(S)S (dithioate), (O)NR₂ (amidate), P(O)R, P(O)OR′, COCH₂ (formacetal), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, the term a “protein” or “polypeptide” may refer to a composition comprised of amino acids and recognized as a protein by those of skill in the art. The conventional one-letter or three-letter code for amino acid residues is used herein. The terms protein and polypeptide are used interchangeably herein to refer to polymers of amino acids of any length, including those comprising linked (e.g., fused) peptides/polypeptides (e.g., fusion proteins). The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, related proteins, polypeptides or peptides may encompass variant proteins, polypeptides, or peptides. Variant proteins, polypeptides or peptides differ from a parent protein, polypeptide, or peptide and/or from one another by a small number of amino acid residues. In some embodiments, the number of different amino acid residues is any of about 1, 2, 3, 4, 5, 10, 20, 25, 30, 35, 40, 45, or 50. In some embodiments, variants differ by about 1 to about 10 amino acids. Alternatively or additionally, variants may have a specified degree of sequence identity with a reference protein or nucleic acid, e.g., as determined using a sequence alignment tool, such as BLAST, ALIGN, and CLUSTAL (see, infra). For example, variant proteins or nucleic acid may have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence.

As used herein, the term “recovered,” “isolated,” “purified,” and “separated” may refer to a material (e.g., a protein, peptide, nucleic acid, polynucleotide or cell) that is removed from at least one component with which it is naturally associated. For example, these terms may refer to a material which is substantially or essentially free from components which normally accompany it as found in its native state, such as, for example, an intact biological system.

As used herein, the term “recombinant” may refer to nucleic acid sequences or polynucleotides, polypeptides or proteins, and cells based thereon, that have been manipulated by man such that they are not the same as nucleic acids, polypeptides, and cells as found in nature. Recombinant may also refer to genetic material (e.g., nucleic acid sequences or polynucleotides, the polypeptides or proteins they encode, and vectors and cells comprising such nucleic acid sequences or polynucleotides) that has been modified to alter its sequence or expression characteristics, such as by mutating the coding sequence to produce an altered polypeptide, fusing the coding sequence to that of another coding sequence or gene, placing a gene under the control of a different promoter, expressing a gene in a heterologous organism, expressing a gene at decreased or elevated levels, expressing a gene conditionally or constitutively in manners different from its natural expression profile, and the like.

As used herein, the term “transfection” or “transformation” may refer to the insertion of an exogenous nucleic acid or polynucleotide into a host cell. The exogenous nucleic acid or polynucleotide may be maintained as a non-integrated vector, for example, a plasmid, or alternatively, may be integrated into the host cell genome. The term transfecting or transfection is intended to encompass all conventional techniques for introducing nucleic acid or polynucleotide into host cells. Examples of transfection techniques include, but are not limited to, calcium phosphate precipitation, diethylaminocthyl (DEAE)-dextran-mediated transfection, lipofection, electroporation, and microinjection.

As used herein, the term “transformed,” “stably transformed,” and “transgenic” may refer to a cell that has a non-native (e.g., heterologous) nucleic acid sequence or polynucleotide sequence integrated into its genome or as an episomal plasmid that is maintained through multiple generations.

As used herein, the term “vector” may refer to a polynucleotide sequence designed to introduce nucleic acids into one or more cell types. Vectors include cloning vectors, expression vectors, shuttle vectors, plasmids, phage particles, single and double stranded cassettes and the like.

As used herein, the term “wild-type,” “native,” or “naturally-occurring” proteins may refer to those proteins found in nature. The terms wild-type sequence refers to an amino acid or nucleic acid sequence that is found in nature or naturally occurring. In some embodiments, a wild-type sequence is the starting point of a protein engineering project, for example, production of variant proteins.

As used herein, the term “non-toxic concentrations” may refer to concentrations of a co-product that have no effect or only a minimal effect on the level of ethanol produced by a yeast modified to produce the co-product compared to the level of ethanol produced by an otherwise similar unmodified yeast. For example, when non-toxic concentrations are present, the level of ethanol produced by the modified yeast may be reduced by no more than 30%, 20%, or, most preferably, no more than 10% compared to the level of ethanol produced by an unmodified yeast.

Unless defined otherwise herein, all technical and scientific terms used herein have the same meaning as commonly understood by one of ordinary skill in the art to which this disclosure belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this disclosure. Further, it will be understood that any of the substrates disclosed in any of the pathways herein may alternatively include the anion or the cation of the substrate.

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Use of the term “about” or “approximately” when referring to a number or a numerical range means that the number or numerical range referred to is an approximation within experimental variability (or within statistical experimental error), and thus the number or numerical range may vary from, for example, between 1% and 15% of the stated number or numerical range. Numeric ranges provided herein are inclusive of the numbers defining the range. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

### Methods for Dehydration of Ethanol and C₃ Alcohol(s)

In one aspect, the disclosure provides a process for production of ethylene and propylene, the process comprising: (a) contacting a mixture of ethanol and at least one C₃ alcohol with a dehydration catalyst in a dehydration unit at a temperature of 250° C. to 500° C. and at a total pressure of 2 bar to 20 bar to produce ethylene and propylene; (b) withdrawing from said dehydration unit a stream containing ethylene and propylene produced from step (a); and (c) separating the ethylene and the propylene from the stream obtained from step (b); wherein the mixture of ethanol and at least one C₃ alcohol has a water content of 0 to 75% by weight of the total weight of the mixture, wherein the mixture is produced by fermentation using a recombinant yeast in one or more fermenters, and wherein the mixture contains at least 50% ethanol by weight of the total weight of ethanol and C₃ alcohols.

In an embodiment, the at least one C₃ alcohol is 1-propanol and/or isopropanol.

In an embodiment, the mixture of ethanol and at least one C₃ alcohol has a water content of 0 to 75% by weight of the total weight of the mixture, such as 0 to 70%, 0 to 65%, 0 to 60%, 0 to 55%, 0 to 50%, 0 to 45%, 0 to 40%, 0 to 35%, 0 to 30%, 0 to 25%, 0 to 20%, 0 to 15%, 0 to 10%, 0 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, or 70 to 75% by weight of the total weight of the mixture. In an embodiment, the mixture of ethanol and at least one C₃ alcohol has a water content of 0 to 75% by weight of the total weight of the mixture. In an embodiment, the mixture of ethanol and at least one C₃ alcohol has a water content of 0 to 25% by weight of the total weight of the mixture.

In an embodiment, the process further comprises adding water to the mixture of ethanol and at least one C₃ alcohol in the dehydration unit to obtain a mixture having a water content of at least 5% by weight of the total weight of the mixture, such as 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, or 70 to 75% by weight of the total weight of the mixture. In an embodiment, the process further comprises adding water to the mixture of ethanol and at least one C₃ alcohol in the dehydration unit to obtain a mixture having a water content of 75% by weight of the total weight of the mixture. In an embodiment, the process further comprises adding water to the mixture of ethanol and at least one C₃ alcohol in the dehydration unit to obtain a mixture having a water content of 25% by weight of the total weight of the mixture. In an embodiment, no water is added to the mixture of ethanol and at least one C₃ alcohol in the dehydration unit.

In an embodiment, the mixture of ethanol and at least one C₃ alcohol contains at least 50% ethanol by weight of the total weight of ethanol and C₃ alcohols, such as at least 60%, at least 70%, at least 80%, at least 90%, at least 95%, 50 to 95%, 50 to 90%, 60 to 90%, and/or 70 to 90% by weight of the total weight of ethanol and C₃ alcohols. In an embodiment, the mixture of ethanol and at least one C₃ alcohol contains at least 50% ethanol by weight of the total weight of ethanol and C₃ alcohols. In an embodiment, the mixture of ethanol and at least one C₃ alcohol contains at least 70% ethanol by weight of the total weight of ethanol and C₃ alcohols.

In an embodiment, the mixture of ethanol and at least one C₃ alcohol further comprises one or more of acetone, salts, heavy components, solids, and other contaminants.

In an embodiment, the dehydration unit comprises two or more reactors, such as 2 to 10, 2 to 9, 2 to 8, 2 to 7, 2 to 6, 2 to 5, 2 to 4, 2, 3, 4, 5, 6, 7, 8, 9, and/or 10 reactors. In an embodiment, the reactors are positioned in series. In an embodiment, the reactors are positioned in parallel. In an embodiment, the reactors are positioned in series and in parallel. In an embodiment, the dehydration unit consists of two or more reactors which are positioned in series and/or in parallel. In an embodiment, the two or more reactors are adiabatic. In an embodiment, the two or more reactors are isothermal. In an embodiment, at least one of the two or more reactors is adiabatic and at least another of the two or more reactors is isothermal.

In an embodiment, the contacting in the dehydration unit is at a temperature of 250° C. to 500° C., such as 250° C. to 480° C., 300° C. to 500° C., 350° C. to 480° C., 400° C. to 480° C., 250° C. to 300° C., 300° C. to 350° C., 350° C. to 400° C., 400° C. to 450° C., and/or 450° C. to 500° C. In an embodiment, the contacting in the dehydration unit is at a temperature of 250° C. to 500° C. In an embodiment, the contacting in the dehydration unit is at a temperature of 350° C. to 480° C. In an embodiment, the contacting in the dehydration unit is at a temperature of 250° C. to 300° C.

In an embodiment, the contacting in the dehydration unit is at a total pressure of 2 bar to 20 bar, such as 5 bar to 20 bar, 5 bar to 15 bar, 8 bar to 12 bar, 2 bar to 5 bar, 5 bar to 10 bar, 10 bar to 15 bar, and/or 15 bar to 20 bar. In an embodiment, the contacting in the dehydration unit is at a total pressure of 2 bar to 20 bar. In an embodiment, the contacting in the dehydration unit is at a total pressure of 5 bar to 15 bar.

In an embodiment, the contacting in the dehydration unit is at a temperature of 350° C. to 480° C. and at a total pressure of 5 bar to 15 bar.

In an embodiment, the dehydration catalyst is selected from zeolites, oxides, heteropolyacids, or a mixture thereof. In an embodiment, the zeolite catalyst is selected from ZSM-5, MOR, or FER. In an embodiment, the heteropolyacid catalyst is selected from Ag₃PW₁₂O₄₀, K₂HPW₁₂O₄₀, or Cs₃PMo₁₂O₄₀. In an embodiment, the oxide catalyst is selected from gamma alumina, eta alumina, or chi alumina. In an embodiment, the gamma alumina has a surface area of 90 m²/g to 200 m²/g, pore volume of 0.45 to 0.70 cm³/g and average pore size width of 9 Angstroms to 11 Angstroms. In an embodiment, the gamma alumina has a surface area of 90 m²/g to 170 m²/g, pore volume of 0.45 to 0.55 cm³/g and average pore size width of 9 Angstroms to 11 Angstroms. In another embodiment, the gamma alumina has a surface area of 350 m²/g to 450 m²/g, pore volume of 1.00 to 1.20 cm³/g and average pore size width of 20 Angstroms to 30 Angstroms.

In an embodiment, the gamma alumina has an acidity measured by an NH₃-TPD test, wherein the gamma alumina has an acid strength distribution of: about 20%-70% weak acid sites, such as about 30-50%, about 30-40%, or about 35-40%. In another embodiment, the gamma alumina has an acidity measured by an NH₃-TPD test, wherein the gamma alumina has an acid site density ranging from about 250 to about 800 μmol/g, preferably from about 300 to about 800 μmol/g, preferably from about 400 to about 800 μmol/g, or preferably from about 500 to about 800 μmol/g.

In an embodiment, the disclosure provides a process for production of ethylene and propylene, the process comprising: (a) contacting a mixture of ethanol and isopropanol with a dehydration catalyst in a dehydration unit at a temperature of 250° C. to 500° C. and a total pressure of 2 bar to 20 bar to produce ethylene and propylene; (b) withdrawing from said dehydration unit a stream containing ethylene and propylene produced from step (a); and (c) separating the ethylene and the propylene from the stream obtained from step (b); wherein the mixture of ethanol and isopropanol has a water content of 0 to 75% by weight of the total weight of the mixture, wherein the mixture is produced by fermentation using a recombinant yeast in one or more fermenters, and wherein the mixture contains at least 70% ethanol by weight of the total weight of ethanol and isopropanol.

In an embodiment, the disclosure provides a process for production of ethylene and propylene, the process comprising: (a) contacting a mixture of ethanol and 1-propanol with a dehydration catalyst in a dehydration unit at a temperature of 250° C. to 500° C. and a total pressure of 2 bar to 20 bar to produce ethylene and propylene; (b) withdrawing from said dehydration unit a stream containing ethylene and propylene produced from step (a); and (c) separating the ethylene and the propylene from the stream obtained from step (b); wherein the mixture of ethanol and 1-propanol has a water content of 0 to 75% by weight of the total weight of the mixture, wherein the mixture is produced by fermentation using a recombinant yeast in one or more fermenters, and wherein the mixture contains at least 70% ethanol by weight of the total weight of ethanol and 1-propanol.

In an embodiment, the disclosure provides a process for production of ethylene and propylene, the process comprising: (a) contacting a mixture of ethanol, isopropanol and 1-propanol with a dehydration catalyst in a dehydration unit at a temperature of 250° C. to 500° C. and a total pressure of 2 bar to 20 bar to produce ethylene and propylene; (b) withdrawing from said dehydration unit a stream containing ethylene and propylene produced from step (a); and (c) separating the ethylene and the propylene from the stream obtained from step (b); wherein the mixture of ethanol, isopropanol and 1-propanol has a water content of 0 to 75% by weight of the total weight of the mixture, wherein the mixture is produced by fermentation using a recombinant yeast in one or more fermenters, and wherein the mixture contains at least 70% ethanol by weight of the total weight of ethanol, isopropanol and 1-propanol.

In an embodiment, the ethylene and the propylene are separated by distillation, filtration, liquid-liquid separation, or a combination thereof.

### Modified Yeast

A yeast may be modified (e.g., genetically engineered) by any method known in the art to comprise and/or express one or more polynucleotides coding for enzymes in a pathway that catalyze a conversion of a fermentable carbon source to one or more products, such as one or more alcohols.

In some embodiments, a yeast may be modified (e.g., genetically engineered) by any method known in the art to comprise and/or express one or more polynucleotides coding for enzymes in a pathway that catalyze a conversion of a fermentable carbon source to intermediates in a pathway for the production of alcohols such as 1-propanol and/or 2-propanol. Such enzymes may include, but are not limited to, any of those enzymes as described herein. For example, the yeast may be modified to comprise one or more polynucleotides coding for enzymes that catalyze a conversion of succinyl-CoA to 1-propanol.

In some embodiments, the yeast may comprise one or more exogenous polynucleotides encoding one or more enzymes in pathways for the production of the alcohols, such as 1-propanol and/or 2-propanol, from a fermentable carbon source.

In one embodiment, the modified yeast is an ethanol-producing yeast which has been modified to co-produce at least one C₃ alcohol. In some embodiments of each or any of the above or below mentioned embodiments, the ethanol-producing yeast is Saccharomyces cerevisiae. In some embodiments of each or any of the above or below mentioned embodiments, the Saccharomyces cerevisiae is an industrial strain. Suitable industrial ethanol producer strains include, but are not limited to, the S. cerevisiae PE-2, CAT-1, and Red strains. In some embodiments of each or any of the above or below mentioned embodiments, the Saccharomyces cerevisiae is any common strain used in ethanol industry, a typical laboratory strain, or any strain resulting from the typical method of crossing between strains. In some embodiments of each or any of the above or below mentioned embodiments, the Saccharomyces cerevisiae is an industrial strain already used in existing industrial ethanol processes, wherein such processes are based on sugar cane, sugar beets, or most preferably, corn as a raw material.

In some embodiments of each or any of the above or below mentioned embodiments, the ethanol-producing yeast is modified to express exogenous phosphoketolase to redirect part of the carbon flow from a renewable raw material (e.g., glucose) to intermediates (e.g., acetate, acetyl-CoA) and therefore to the at least one C₃ alcohol of interest (e.g., isopropanol) through the pentose phosphate pathway (PPP).

In some embodiments of each or any of the above or below mentioned embodiments, the ethanol-producing yeast is modified to express exogenous phosphoenolpyruvate carboxykinase (PEPCK) to redirect carbon flow from PEP to oxaloacetate. In some embodiments of each or any of the above or below mentioned embodiments, the ethanol-producing yeast is modified to express a PEPCK to redirect the carbon flow from a renewable raw material (e.g., glucose) to intermediates (e.g., oxaloacetate, malonate semialdehyde) and therefore to the at least one C₃ alcohol of interest (e.g., 1-propanol, isopropanol).

In some embodiments of each or any of the above or below mentioned embodiments, the ethanol-producing yeast is modified to knock-out endogenous genes and/or downregulate endogenous enzymes including, but not limited to, pyruvate decarboxylase (e.g., PDC1), pyruvate kinase (e.g., PYK1), and alcohol dehydrogenases. In some embodiments of each or any of the above or below mentioned embodiments, the downregulation of endogenous genes is carried out by a weak promoter (either natural or synthetic), natural or synthetic terminators, natural or synthetic transcription factors, degron peptides, iCRISPR, or any other technique known in the art for downregulation of genes in yeast. In some embodiments of each or any of the above or below mentioned embodiments, the weak promoter is pADH1, pCYC1, pSTE5, PREV1, PURA3, pRPLA1, pGAP1, pNUP57, or pMET25.

In some embodiments of each or any of the above or below mentioned embodiments, the C₃ alcohols are produced at non-toxic concentrations for the ethanol-producing yeast. In some embodiments of each or any of the above or below embodiments, the engineered ethanol-producing yeast was genetically modified to co-produce the at least one C₃ alcohol of interest (e.g., 1-propanol, 2-propanol, or a combination thereof) along with ethanol as the major product in the fermentation broth (see, e.g., U.S. Patent Application Publication No. 2021/0261987).

In some embodiments of each or any of the above or below mentioned embodiments, the engineered ethanol-producing yeast modified to produce the at least one C₃ alcohol of interest (e.g., isopropanol, 1-propanol) remains the ethanol fermentation robustness and performance required for the industrial deployment. In some embodiments of each or any of the above or below mentioned embodiments, the recombinant yeast has most of the ethanol fermentation robustness and performance preserved compared to its mother industrial ethanol-producing yeast, enabling its use on already existing industrial ethanol processes.

A modified yeast as provided herein may comprise:
one or more polynucleotides coding for enzymes in a pathway that catalyzes a conversion of a fermentable carbon source to succinyl-CoA,
one or more polynucleotides coding for enzymes in a pathway that catalyzes a conversion of a fermentable carbon source to 1,2-propanediol,
one or more polynucleotides coding for enzymes in a pathway that catalyzes a conversion of a fermentable carbon source to lactate,
one or more polynucleotides coding for enzymes in a pathway that catalyzes a conversion of a fermentable carbon source to β-alanine,
one or more polynucleotides coding for enzymes in a pathway that catalyzes a conversion of a fermentable carbon source to threonine,
one or more polynucleotides coding for enzymes in a pathway that catalyzes a conversion of a fermentable carbon source to citramalate,
one or more polynucleotides coding for enzymes in a pathway that catalyzes a conversion of fermentable carbon source to malonate semialdehyde,
one or more polynucleotides coding for enzymes in a pathway that catalyzes a conversion of succinyl-CoA to methylmalonyl-CoA,
one or more polynucleotides coding for enzymes in a pathway that catalyzes a conversion of threonine to 2-ketobutyrate (2-κB),
one or more polynucleotides coding for enzymes in a pathway that catalyzes a conversion of citramalate to 2-ketobutyrate (2-κB),
one or more polynucleotides coding for enzymes in a pathway that catalyzes a conversion of β-alanine to malonate semialdehyde,
one or more polynucleotides coding for enzymes in a pathway that catalyzes a conversion of malonate semialdehyde to 3-hydroxypropionate (3-HP),
one or more polynucleotides coding for enzymes in a pathway that catalyzes a conversion of lactate to acrylyl-CoA,
one or more polynucleotides coding for enzymes in a pathway that catalyzes a conversion of β-alanine to acrylyl-CoA,
one or more polynucleotides coding for enzymes in a pathway that catalyzes a conversion of 3-HP to acrylyl-CoA,
one or more polynucleotides coding for enzymes in a pathway that catalyzes a conversion of methylmalonyl-CoA to propionyl-CoA,
one or more polynucleotides coding for enzymes in a pathway that catalyzes a conversion of 2-kB to propionyl-CoA,
one or more polynucleotides coding for enzymes in a pathway that catalyzes a conversion of acrylyl-CoA to propionyl-CoA,
one or more polynucleotides coding for enzymes in a pathway that catalyzes a conversion of propionyl-CoA to propionaldehyde,
one or more polynucleotides coding for enzymes in a pathway that catalyzes a conversion of 1,2-propanediol to propionaldehyde, and/or
one or more polynucleotides coding for enzymes in a pathway that catalyzes a conversion of propionaldehyde to 1-propanol.

A modified microorganism as provided herein may comprise:
one or more polynucleotides coding for enzymes in a pathway that catalyzes a conversion of fermentable carbon source to pyruvate,
one or more polynucleotides coding for enzymes in a pathway that catalyzes a conversion of fermentable carbon source to malonate semialdehyde (MSA),
one or more polynucleotides coding for enzymes in a pathway that catalyzes a conversion of pyruvate to acetyl-CoA,
one or more polynucleotides coding for enzymes in a pathway that catalyzes a conversion of MSA to acetyl-CoA;
one or more polynucleotides coding for enzymes in a pathway that catalyzes a conversion of acetyl-CoA to acetoacetyl-CoA,
one or more polynucleotides coding for enzymes in a pathway that catalyzes a conversion of acetyl-CoA to malonyl-CoA,
one or more polynucleotides coding for enzymes in a pathway that catalyzes a conversion of malonyl-CoA to acetoacetyl-CoA,
one or more polynucleotides coding for enzymes in a pathway that catalyzes a conversion of acetoacetyl-CoA to acetoacetate,
one or more polynucleotides coding for enzymes in a pathway that catalyzes a conversion of acetoacetyl-CoA to hydroxymethylglutaryl-CoA (HMG-COA),
one or more polynucleotides coding for enzymes in a pathway that catalyzes a conversion of HMG-COA to acetoacetate,
one or more polynucleotides coding for enzymes in a pathway that catalyzes a conversion of acetoacetate to acetone, and/or
one or more polynucleotides coding for enzymes in a pathway that catalyzes a conversion of acetone to 2-propanol.

A modified microorganism as provided herein may comprise:
one or more polynucleotides coding for enzyme that catalyzes a conversion of fermentable carbon source to acetyl-CoA through the pentose phosphate pathway (PPP),
one or more polynucleotides coding for enzymes in a pathway that catalyzes a conversion of acetyl-CoA to acetoacetyl-CoA,
one or more polynucleotides coding for enzymes in a pathway that catalyzes a conversion of acetoacetyl-CoA to acetoacetate,
one or more polynucleotides coding for enzymes in a pathway that catalyzes a conversion of acetoacetate to acetone, and/or
one or more polynucleotides coding for enzymes in a pathway that catalyzes a conversion of acetone to 2-propanol.

In some embodiments, the yeast is Saccharomyces cerevisiae, Kluyveromyces lactis or Pichia pastoris.

In some embodiments, the yeast is Saccharomyces cerevisiae and is an industrial ethanol producer yeast, i.e., a yeast strain already used in existing industrial ethanol fermentation processes and assets, wherein such industrial yeast has appropriate and distinguished robustness and fermentation performance to the production of ethanol.

In some embodiments, the yeast is Saccharomyces cerevisiae and is an industrial ethanol producer yeast already used in existing industrial ethanol fermentation processes and assets, wherein such processes and assets are based on sugar cane, sugar beets or corn as a raw material.

In some embodiments, the yeast is Saccharomyces cerevisiae and is an industrial ethanol producer yeast derived from or industrially used in already existing corn-based ethanol fermentation processes and assets.

In some embodiments, the yeast is additionally modified to comprise one or more tolerance mechanisms including, for example, tolerance to a produced molecule (e.g., 1-propanol and/or 2-propanol), and/or organic solvents. A yeast modified to comprise such a tolerance mechanism may provide a means to increase titers of fermentations and/or may control contamination in an industrial scale process.

Host cells are transformed or transfected with the expression or cloning vectors disclosed herein for production of one or more enzymes as disclosed herein or with polynucleotides coding for one or more enzymes as disclosed herein and cultured in conventional nutrient media modified as appropriate for inducing promoters, selecting transformants, or amplifying the genes encoding the desired sequences.

Host cells containing desired nucleic acid sequences coding for the disclosed enzymes may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44, (1979); Barnes et al., Anal. Biochem. 102:255 (1980); U.S. Pat. Nos. 4,767,704; 4,657,866; 4,927,762; 4,560,655; or 5,122,469; WO 90/103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Pathways for Production of 1-Propanol

Metabolic pathways for the production of 1-propanol include pathways that produce 1-propanol from intermediates including, but not limited to, malonate semialdehyde, 3-hydroxypropionic acid, 1,2-propanediol, 2-ketobutyrate (2-kB), succinyl-CoA, and acrylyl-CoA. Specifically, the 2-kB, succinyl-CoA, and acrylyl-CoA intermediates converge into propionyl-CoA. Both propionyl-CoA and 1,2-propanediol are converted to propionaldehyde and to 1-propanol by a bi-functional aldehyde/alcohol dehydrogenase or by the action of an aldehyde dehydrogenase (acetylating) in combination with an alcohol dehydrogenase.

In one pathway, 1-propanol is produced via the succinyl-CoA route whereby a sugar source is converted to succinyl-CoA via glycolysis and the citric acid cycle (TCA cycle), followed by the isomerization of succinyl-CoA to methylmalonyl-CoA by a methylmalonyl-CoA mutase, and the decarboxylation of methylmalonyl-CoA to propionyl-CoA by a methylmalonyl-CoA decarboxylase. Aldehyde and alcohol dehydrogenases catalyze additional conversions to convert propionyl-CoA to propionaldehyde and propionaldehyde to 1-propanol (see, e.g., U.S. Patent Application Publication No. 2013/0280775). In another pathway, 1-propanol is produced via 1,2-propanediol whereby a sugar source undergoes multiple conversions catalyzed by a methylglyoxal synthase, an aldo-ketoreductase or a glyoxylate reductase and an aldehyde reductase. Hydrolase and dehydrogenases catalyze additional conversions to convert 1,2-propanediol to propanal and propanal to 1-propanol (see, e.g., U.S. Pat. No. 9,957,530).

In another pathway, 1-propanol is produced from a 2-kB intermediate via conversions from threonine and/or citramalate. For example, 2-kB can be converted to propionyl-CoA or directly to propionaldehyde by a 2-oxobutanoate dehydrogenase or a 2-oxobutanoate decarboxylase, respectively (see, e.g., U.S. Patent Application Publication No. 2014/0377820).

In other pathways, 1-propanol is produced from β-alanine, oxaloacetate, lactate, or 3-hydroxypropionate (3-HP) intermediates that converge to acrylyl-CoA, which is converted to propionyl-CoA by an acrylyl-CoA reductase (see, e.g., U.S. Patent Application Publication No. 2014/0377820). As described above, propionyl-CoA can be converted to 1-propanol by aldehyde and alcohol dehydrogenases.

### Pathways for Production of 2-Propanol

Metabolic pathways for the production of 2-propanol include pathways that produce 2-propanol from intermediates including, but not limited to, propane and/or acetone. Acetone can be generated from several pathways, including but not limited to primary and secondary metabolism reactions, as glycolysis, terpenoid biosynthesis, atrazine degradation and cyanoamino acid metabolism. In one pathway, acetyl-CoA can be derived from pyruvate and/or malonate semialdehyde by a pyruvate dehydrogenase and a malonate semialdehyde dehydrogenase, respectively. Acetyl-CoA is converted to acetoacetyl-CoA by a thiolase or an acetyl-CoA acetyltransferase (see, e.g., U.S. Patent Application Publication No. 2018/0179558). Alternatively, acetoacetyl-CoA can be formed through malonyl-CoA by acetoacetyl-CoA synthase. Once acetoacetyl-CoA is formed, its conversion to acetoacetate can be done by an acetoacetyl-CoA transferase or through HMG-COA by hydroxymethylglutaryl-CoA synthase and hydroxymethylglutaryl-CoA lyase. Acetoacetate conversion to acetone is done by an acetoacetate decarboxylase.

In another pathway, 2-propanol is produced from propane and/or acetone as precursors. As described above, acetone is generated from acetyl-CoA by multiple reactions and is converted to isopropanol by an isopropanol dehydrogenase (see, e.g., U.S. Patent Application Publication No. 2018/0179558). In another pathway, propane is produced from a butyrate intermediate and isopropanol is generated by a propane 2-monooxygenase. Biosynthesis of propane in Escherichia coli from glucose having butyrate as intermediate is described in Kallio et al. (2014) Nat Commun, 5 (4731).

In another pathway, 2-propanol (isopropanol) is produced from acetone as a precursor. In one embodiment, isopropanol is produced through a metabolic pathway comprising one or more polynucleotide coding for enzymes with phosphoketolase activity that enables the conversion of a renewable carbon source (e.g., glucose) into intermediates (e.g., acetyl-CoA) and further into acetone and/or isopropanol. In one embodiment, the engineered metabolic pathway is the pentose phosphate pathway (PPP). In one embodiment, the engineered metabolic pathway comprises the conversion of D-xylulose 5-phosphate to D-glyceraldehyde 3-phosphate and acetyl phosphate, or the conversion of D-fructose 6-phosphate to D-erythrose 4-phosphate and acetyl phosphate. In some embodiments, D-xylulose 5-phosphate is converted to D-glyceraldehyde 3-phosphate and acetyl phosphate by a phosphoketolase. In some embodiments, D-fructose 6-phosphate is converted to D-erythrose 4-phosphate and acetyl phosphate by a phosphoketolase. Phosphoketolases include enzymes classified as (EC) 4.1.2.9 and (EC) 4.1.2.22. In one embodiment, phosphoketolase is from Bifidobacterium animalis, Bifidobacterium longum, Bifidobacterium adolescentis, Bifidobacterium breve, Aspergillus nidulans, Aspergillus niger, Clostridium acetobutylicum, Leuconostoc mesenteroides, Lactobacillus plantarum, Lactobacillus casei, Lactobacillus pentosum and Lactobacillus acidophilus. In some embodiments, the engineered metabolic pathway comprises the conversion of acetyl phosphate to acetyl-CoA by one or more native and/or heterologous acyltransferases. Acetyltransferases include enzymes classified as (EC) 2.3.1.8. In some embodiments, the engineered metabolic pathway comprises the conversion of acetyl phosphate to acetate by one or more native and/or heterologous phosphatases or phosphotransferases (PTA). Phosphotransferases include enzymes classified as (EC) 2.7.2.1. In some embodiments, the engineered metabolic pathway comprises the conversion of acetate to acetyl-CoA by a native and/or heterologous acetyl-CoA synthetase (ACS). Acetyl-CoA synthetases include enzymes classified as (EC) 6.2.1.1. In yet another embodiment, the modified yeast comprises a combination of enzymes to convert the acetyl-CoA into acetoacetyl-CoA, acetoacetate, acetone and/or isopropanol.

### Method for the Co-Production of Ethanol and C₃ Alcohols

In an embodiment, ethanol and at least one C₃ alcohol are produced by a process comprising: contacting a fermentable carbon source with an ethanol-producing yeast in a fermentation medium; and fermenting the carbon source by the yeast in the fermentation medium to produce a fermentation broth and a fermentation off-gas comprising ethanol and the at least one C₃ alcohol.

In one embodiment, the fermentable carbon source is contacted with an ethanol-producing yeast comprising one or more polynucleotides coding for enzymes in a pathway that catalyzes a conversion of the fermentable carbon source to any of the intermediates in the production of the at least one C₃ alcohol and one or more polynucleotides coding for enzymes in a pathway that catalyze a conversion of the one or more intermediates to the at least one C₃ alcohol in a fermentation media; and expressing the one or more polynucleotides coding for the enzymes in the pathway that catalyzes a conversion of the fermentable carbon source to the one or more intermediates in the production of the at least one C₃ alcohol and one or more polynucleotides coding for enzymes in a pathway that catalyze a conversion of the one or more intermediates to the at least one C₃ alcohol. In one embodiment, ethanol and the at least one C₃ alcohol are produced by contacting any of the genetically modified yeasts provided herein with the fermentable carbon source. In one embodiment, the ethanol-producing yeast is a genetically modified Saccharomyces cerevisiae.

In one embodiment, the fermentation products of the disclosure may be prepared by conventional processes for industrial sugar cane, wheat, cassava, sweet sorghum, sugar beet, beet, or corn ethanol production. In one embodiment, the fermentable carbon source comprises a C₅, C₆, or C₁₂ sugar. In one embodiment, the C₅, C₆, or C₁₂ sugar originates from a renewable source that is already used in a conventional ethanol mill. Therefore, in some embodiments, the C₅, C₆, or C₁₂ sugar source is sugar cane, corn, wheat, cassava, sweet sorghum, sugar beets, beets, cellulose, biomass, or biomass waste. In one embodiment, the sugar is a C₆ sugar. In one embodiment, the C₆ sugar is glucose. In one embodiment, the at least one C₃ alcohol is 1-propanol and/or 2-propanol.

In one embodiment, an aqueous stream comprising the C₅, C₆, or C₁₂ sugar fermentable carbon source enters a fermentation unit and is contacted with the ethanol-producing yeast. In one embodiment, a solution comprising the C₅, C₆, or C₁₂ sugar enters the fermenter by a renewable feedstock pipe. In one embodiment, the C₅, C₆, or C₁₂ sugar solution comprises from about 65% w/w to about 85% w/w of water, about 10% w/w to about 25% w/w of C₅, C₆, or C₁₂ sugar, about 1% w/w to about 3.5% w/w of celluloses, and about 0% to about 5% w/w of residual biomass, depending on the feedstock. In one embodiment, the C₅, C₆, or C₁₂ sugar solution comprises about 75% w/w water, about 18% w/w of C₅, C₆, or C₁₂ sugar, about 2.4% w/w celluloses, and about 0% to 5% w/w of residual biomass. In one embodiment, the sugar is a C₆ sugar. In one embodiment, the C₆ sugar is glucose. In one embodiment, the fermentation media may additionally contain suitable minerals, salts, cofactors, buffers and other components suitable for the growth and maintenance of the cultures.

In one embodiment, the mixture of genetically modified yeast and the C₅, C₆, or C₁₂ sugar undergoes a fermentation step, wherein the genetically modified yeast produces the target C₃ alcohol(s) such as 1-propanol and 2-propanol along with ethanol. In one embodiment, the fermentation step is carried out either in accordance with a batch operation mode, a multi-stage batch operation mode, a semi-continuous operational mode (or “fed-batch” mode), or in accordance with an operational mode known as a continuous mode; these are well known to the person skilled in the art.

In one embodiment, the fermentation step may be conducted by different engineered yeast strains, keeping the industrial ethanol performance (titer, yield, productivity) similar to what is expected or already obtained by the ethanol millers, all always producing ethanol in larger quantities than the target C₃ alcohol(s). In one embodiment, the fermentation step is carried out at a temperature of about 30° C. to about 37° C. In one embodiment, the process of producing the ethanol and the at least one C₃ alcohol can be implemented in existing industrial ethanol mills with little or ideally no modification in the industrial fermentation area.

In one embodiment, the step of fermentation produces greater than about 90 g/L, about 100 g/L, about 110 g/L, about 120 g/L, about 130 g/L, about 140 g/L, or about 150 g/L of ethanol after fermentation for less than about 72 hours, about 60 hours, about 55 hours, about 50 hours, about 45 hours, or about 40 hours, or about 10 hours, or about 5 hours at an industrial scale. In one embodiment, the step of fermentation produces greater than about 2.5 g/L, about 5 g/L, about 10 g/L, about 15 g/L, about 20 g/L or about 30 g/L of the at least one C₃ alcohol after fermentation for less than about 72 hours, about 60 hours, about 55 hours, about 50 hours, about 45 hours, or about 40 hours, or about 10 hours, or about 5 hours at an industrial scale.

In one embodiment, the fermentation step reaches a high titer of greater than about 110 g/L, about 120 g/L, about 130 g/L, about 140 g/L, about 150 g/L, or about 160 g/L of total solvents. In one embodiment, the fermentation step reaches a high titer of greater than about 140 g/L of total solvents. In one embodiment, the fermentation step reaches a productivity of greater than about 1.0 g/L·h, about 1.5 g/L·h, about 2.0 g/L·h, about 2.5 g/L·h, about 3.0 g/L·h, about 3.5 g/L·h, about 4.0 g/L·h, about 4.5 g/L·h, or about 5.0 g/L·h of solvents. In one embodiment, the term “total solvents” refers to the combination of ethanol and the at least one C₃ alcohol. In one embodiment the fermentation step reaches a productivity of greater than about 2.5 g/L·h of solvents.

In one embodiment, the ethanol and the at least one C₃ alcohol in the fermentation broth is separated by a process described herein.

### Separation of Ethanol and C₃ Alcohol(s)

In an embodiment, the ethanol and at least one C₃ alcohol is isolated from a fermentation broth and a fermentation off-gas by a process comprising: (a) flowing a fermentation off-gas coming from one or more fermenters through a product recovery unit wherein the flow of the off-gas is counter to a solvent flowing through the product recovery unit and obtaining a solvent stream comprising the at least one C₃ alcohol and ethanol; (b) mixing the solvent stream of (a) with the fermentation broth and passing the resulting fermentation mixture through a first separation unit to form a high water content stream, comprising ethanol and the at least one C₃ alcohol; (c) passing the high water content stream of (b) through a second separation unit to form an intermediate water content stream, comprising ethanol and the at least one C₃ alcohol; and (d) passing the ethanol intermediate water content stream of (c) through a dewatering unit to form a low water content stream. In an embodiment, the ethanol and at least one C₃ alcohol is isolated from a fermentation broth and a fermentation off-gas by a process comprising steps (a) to (d) above and the resulting low water content stream of (d) is passed to a dehydration unit.

The isolation process can treat an alcoholic fermentation broth comprising the one or more C₃ alcohols described herein diluted in ethanol and water. In some embodiments, the concentration of the one or more C₃ alcohols in the fermentation broth ranges from about 5 g/L to 30 g/L, and ethanol as the main product from a concentration of about 40 g/L to 140 g/L. In one embodiment, the concentration of the C₃ alcohols and ethanol depends on the feedstock used for the industrial ethanol fermentation processes (e.g. corn, wheat, sugar beets, sugar cane, or other biomass materials). The output of this fermentation process leaves the fermenter either in the off-gas (vapor phase) or in the broth (liquid phase).

In one embodiment, the fermentation off-gas of (a) contains one or more incondensable gases, water, ethanol, and the at least one C₃ alcohol. In one embodiment, the fermentation off-gas comprises between about 80% w/w to about 98% w/w, about 85% w/w to about 98% w/w, about 90% w/w to about 98% w/w, or about 92% w/w to about 98% w/w of an incondensable gas. In one embodiment, the fermentation off-gas comprises between about 0.5% w/w to about 15% w/w, about 0.5% w/w to about 12% w/w, about 0.5% w/w to about 10% w/w, about 1% w/w to about 10% w/w, about 1% w/w to about 8% w/w, about 1% w/w to about 6% w/w, or about 2% w/w to about 5% of ethanol and the at least one C₃ alcohol. In one embodiment, the fermentation off-gas comprises between about 0.01% w/w to about 15% w/w, about 0.01% w/w to about 12% w/w, about 0.01% w/w to about 10% w/w, about 0.01% w/w to about 8% w/w, about 0.01% w/w to about 6% w/w, about 0.1% w/w to about 6% w/w, about 0.1% w/w to about 4% w/w, or about 0.5% w/w to 3% w/w water. In one embodiment, the incondensable gas is primarily carbon dioxide.

In one embodiment, the fermentation broth of (a) comprises water, the at least one C₃ alcohol, ethanol, and one or more contaminants or congeners. In one embodiment, the fermentation broth comprises the at least one C₃ alcohol in a concentration between about 0.5 g/L and about 100 g/L, about 0.5 g/L and about 90 g/L, about 0.5 g/L and about 80 g/L, about 0.5 g/L and about 70 g/L, about 0.5 g/L and about 60 g/L, about 0.5 g/L and about 50 g/L, about 1 g/L and about 40 g/L, or about 5 g/L and about 30 g/L. In one embodiment, ethanol is the main product in the fermentation broth at a concentration of between about 10 g/L and about 200 g/L, about 20 g/L and about 180 g/L, about 30 g/L and about 160 g/L, or about 40 g/L and about 140 g/L. In one embodiment, the fermentation broth comprises less than about 30 g/L, about 25 g/L, about 20 g/L, about 15 g/L, about 10 g/L or about 5 g/L of volatile contaminants or congeners that have to be removed to purify the at least one C₃ alcohol and ethanol. In one embodiment, the congeners were already present in the feedstock or were produced by the fermentation process, and may be organic acids, organic alcohols, and other organic compounds.

In one embodiment, the congeners may include, but are not limited to, aldehydes (e.g. acetaldehyde and acetal), ketones (e.g. diketone, ethyl methyl ketone, and 2-butanone), esters (e.g. ethyl formate, ethyl acetate, propyl acetate, 2-methylpropyl acetate, 3-methylbutyl acetate, ethyl hexanoate, hexyl acetate, ethyl lactate, ethyl isovalerate, ethyl-alpha-methylbutyrate, 2-methylpropyl hexanoate, ethyl lactate, 2-methylpropyl hexanoate, ethyl octanoate, 3-methylbutyl hexanoate, ethyl phenylacetate, phenyl ethyl acetate, ethyl decanoate, ethyl dodecanoate, and ethyl tetradecanoate), alcohols (e.g. methanol, 2-methylbutan-1-ol, 3-methylbutan-1-ol, 1-pentanol,2-phenylethan-1-ol, and glycerol), and organic acids (e.g. acetic acid, lactic acid, propionic acid, i-butyric acid, butyric acid, valeric acid, i-valeric acid, hexanoic acid, octanoic acid, decanoic acid, and dodecanoric acid).

In one embodiment, in the product recovery unit of (a), the products present in the off-gas are absorbed by the solvent (e.g., water) added at the top of the unit. In one embodiment, the product recovery unit is an absorption column or a scrubber column. In one embodiment, between about 0.25 parts to about 4 parts of solvent flow counter to about one part of off-gas on a mass basis. In another embodiment, about 0.5 part to 2 parts solvent flow counter to about one part of off-gas on a mass basis. In one embodiment, the solvent is at a temperature of about 5° C. to about 100° C., about 5° C. to about 90° C., about 5° C. to about 80° C., about 5° C. to about 70° C., about 5° C. to about 60° C., about 5° C. to about 50° C., about 5° C. to about 50° C., or about 5° C. to about 45° C. In one embodiment, the solvent is at a temperature of about 30° C. In one embodiment, the solvent is water.

In one embodiment, step (a) yields a solvent stream comprising the at least one C₃ alcohol and ethanol as well as a purified gas stream. In one embodiment, the purified gas stream comprises more than about 70%, about 75%, about 80%, about 85%, about 90%, or about 95% incondensable gases and less than about 30%, about 25%, about 20%, about 15%, about 10%, about 5%, about 1%, or about 0.5% by weight of ethanol and the at least one C₃ alcohol.

In one embodiment, the fermentation mixture of (b) is stored in a tank. In one embodiment, the fermentation mixture tank is integrated with a stillage heat exchanger and the stillage heat exchanger preheats the fermentation mixture before it is passed through the first separation unit of (b). In one embodiment, the fermentation mixture of (b) is called “beer” and is stored in a beer tank. In one embodiment, the first separation unit is an evaporator, a distillation column, a set of distillation columns, a combination of a centrifuge and a distillation column, or a combination of a centrifuge and a set of distillation columns. In one embodiment, the distillation column is capable of treating solids, usually called beer or mash column. In one embodiment, the high-water content stream of (b) comprises ethanol, the at least one C₃ alcohol, and water. In one embodiment, the high-water content stream comprises between about 10% w/w to about 90% w/w, about 25% w/w to about 90% w/w, about 30% w/w to about 90% w/w, about 30% w/w to about 85% w/w, about 35% w/w to about 85% w/w, or about 35% w/w to 80% w/w of water. In one embodiment, the high-water content stream comprises about 10% w/w to about 90% w/w, about 10% w/w to about 85% w/w, about 10% w/w to about 80% w/w, about 15% w/w to about 70% w/w, about 15% w/w to about 65% w/w, or 20% w/w to 65% w/w of ethanol and the at least one C₃ alcohol. In one embodiment, the high water content stream is maintained at a temperature of between about 40° C. and about 180° C., about 40° C. and about 170° C., about 40° C. and about 160° C., about 50° C. and about 160° C., about 50° C. and about 150° C., about 60° C. and about 150° C., about 60° C. and about 140° C., or about 70° C. to about 135° C.

In one embodiment, if a distillation column is used in (b), the column operates at a pressure of between about 0.1 bar and about 5 bar or about 0.1 bar and about 3 bar. In one embodiment, prior to distillation the beer is heated to a temperature of between about 40° C. and 150° C., about 45° C. and about 145° C., about 50° C. and about 140° C., about 55° C. and about 135° C., about 60° C. and about 130° C., about 65° C. and about 125° C., or about 70° C. and about 115° C. In one embodiment, heat integrations with the distillation column condensers will depend on the products being separated and operating conditions of the other separation units.

In one embodiment, the first separation unit removes an output “stillage” stream comprising water, heavy components, and solids to a stillage tank. In one embodiment, the stillage can have different end uses, according to the feedstock selected, and may have a content of about 55% w/w to about 99% w/w of water, about 60% w/w to about 99% w/w of water, or about 65% w/w to about 99% w/w of water and the rest will be composed of solids present in the feedstock and C₅, C₆, or C₁₂ sugars that were not consumed during fermentation, in the range of about 45% w/w or less. In one embodiment, the stillage stream leaves the first separation unit at a temperature of about 30° C. and about 200° C., about 30° C. and about 190° C., about 30° C. and about 180° C., about 40° C. and about 180° C., about 50° C. and about 180° C., about 50° C. and about 170° C., about 50° C. and about 160° C., about 60° C. and about 160° C., about 60° C. and about 150° C., or about 65° C. and about 140° C.

In one embodiment, the process further comprises recycling a gas stream output from the first separation unit of (b) by passing the gas stream output through steps (a) and (b). In one embodiment, the gas stream output comprises an incondensable gas which is removed as the gas stream is passed through steps (a) and (b) and wherein the gas stream further comprises the at least one C₃ alcohol which are recovered. In one embodiment, the gas stream output comprises between about 20% w/w to about 95% w/w, about 25% w/w to about 95% w/w, about 30% w/w to about 95% w/w, about 35% w/w to about 90% w/w, or about 40% w/w to about 85% w/w incondensable gas. In one embodiment, the gas stream output comprises between about 1% w/w to about 75% w/w, about 1% w/w to about 75% w/w, about 1% w/w to about 75% w/w, about 5% w/w to about 70% w/w, about 5% w/w to about 65% w/w, about 10% w/w to about 65% w/w, or about 15% w/w to about 60% w/w of ethanol and the at least one C₃ alcohol. In one embodiment, the incondensable gas comprises CO₂. In one embodiment, the gas stream leaves the first separation unit at a temperature of about 10° C. and about 160° C., about 15° C. and about 155° C., about 20° C. and about 150° C., about 25° C. and about 145° C., about 30° C. and about 140° C., about 30° C. and about 130° C., about 30° C. and about 120° C., about 30° C. and about 110° C., about 30° C. and about 105° C., or about 30° C. and about 100° C.

In one embodiment, the second separation unit of (c) is a rectifier column, a distillation column, or a set of distillation columns.

In one embodiment, the second separation unit of (c) is a distillation column known as a rectifier column. In one embodiment, the second separation unit removes an output stream comprising primarily water. In one embodiment, the output stream comprises between about 70% w/w to about 100% w/w, about 75% w/w to about 100% w/w, or about 80% w/w to about 100% w/w water. In one embodiment, the output stream is maintained at a temperature of about 40° C. to about 200° C., about 45° C. to about 195° C., about 45° C. to about 185° C., about 45° C. to about 185° C., about 45° C. to about 175° C., about 50° C. to about 170° C., about 50° C. to about 160° C., about 55° C. to about 155° C., about 60° C. to about 150° C., about 60° C. to about 140° C., or about 65° C. to about 135° C. In one embodiment, the process further comprises the step of recycling the water stream output of (c) to an upstream fermentation process which produces the fermentation broth of (a). In one embodiment, this recycling reduces the water consumption of the process described herein. In another embodiment, the process further comprises the step of recycling the bottom output stream of (c) by passing the bottom output stream through step (c). In yet another embodiment, the process further comprises the step of recycling the water stream output of (c) back to (b).

In one embodiment, the second separation unit removes a side stream comprising fusel oil. In one embodiment, the side stream is at a temperature of between about 15° C. and about 180° C., about 20° C. to about 175° C., about 25° C. to about 170° C., about 30° C. to about 165° C., about 35° C. to about 160° C., about 40° C. to about 155° C., about 45° C. to about 150° C., about 45° C. to about 140° C., about 50° C. to about 135° C., about 50° C. to about 125° C., or about 55° C. to about 120° C.

In one embodiment, the intermediate water content stream of (c) comprises ethanol, water, and the at least one C₃ alcohol. In one embodiment, the intermediate water content stream comprises between about 60% w/w to about 95% w/w, about 65% w/w to about 95% w/w, about 70% w/w to about 95% w/w, or about 75% w/w to about 95% w/w ethanol. In one embodiment, the intermediate water content stream further comprises between about 5% w/w to about 30% w/w, about 5 w/w to about 25% w/w, about 5% w/w to about 20% w/w, about 5% w/w to about 15% w/w, or about 5% w/w to about 10% w/w water. In one embodiment, the intermediate water content stream further comprises between about 0.1 w/w to about 25% w/w, about 5% w/w to about 20% w/w, or about 1% w/w to about 15% w/w of the at least one C₃ alcohol. In one embodiment, the intermediate water content stream is maintained at a temperature of between about 15° C. and 180° C., about 20° C. and about 175° C., about 20° C. to about 165° C., about 25° C. to about 160° C., about 30° C. to about 155° C., about 35° C. to about 150° C., about 35° C. to about 140° C., about 40° C. to about 135° C., about 40° C. to about 125° C., about 45° C. to about 115° C., or about 50° C. to about 110° C. In one embodiment, the rectifier operates at a pressure of between about 0.1 bar to about 5 bar or about 0.1 bar to about 3 bar.

In one embodiment, the process further comprises recycling a vent gas stream output from the rectifier column of (c) by passing the vent gas stream output through step (a) to reduce product loss.

In one embodiment, the process further comprises removing a top vapor stream from the second separation unit of (c) and recycling the top vapor stream by combining the top vapor stream with the fermentation off-gas. In one embodiment, the process further comprises passing the combined stream comprising the top vapor stream and the fermentation off-gas through step (a). In another embodiment, the process further comprises passing the combined stream comprising the top vapor stream and the fermentation off-gas through steps (a) and (b). In yet another embodiment, the process further comprises passing the combined stream comprising the top vapor stream and the fermentation off-gas through steps (a), (b), and (c).

In one embodiment, the dewatering unit of (d) is a membrane separation system or a molecular sieve system. In one embodiment, the low water content stream of (d) comprises between about 15% w/w and 0.5%, about 10% w/w and 0.5% w/w, about 5% w/w and 0.5% w/w, or about 2.5% w/w and 0.5% w/w water. Although not wishing to be limited by theory, it is believed that the presence of the dewatering step before the dehydration of the ethanol and the at least one C₃ alcohol is key to the operating expense reduction, the preservation of product specification, and the economic viability of the process described herein. In one embodiment, the process further comprises the step of recycling a water stream output from the dewatering unit of (d) by passing the water stream output through steps (c) and (d). In some embodiments, recycling the water stream output through steps (c) and (d) functions to recover any desired ethanol or at least one C₃ alcohol which are present in the water stream output.

In one embodiment, the process further comprises removing a top vapor stream from the dewatering unit of (d) and recycling the top vapor stream by combining the top vapor stream with the fermentation off-gas. In one embodiment, the process further comprises passing the combined stream comprising the top vapor stream and the fermentation off-gas through step (a). In another embodiment, the process further comprises passing the combined stream comprising the top vapor stream and the fermentation off-gas through steps (a) and (b). In another embodiment, the process further comprises passing the combined stream comprising the top vapor stream and the fermentation off-gas through steps (a), (b), and (c). In yet another embodiment, the process further comprises passing the combined stream comprising the top vapor stream and the fermentation off-gas through steps (a), (b), (c), and (d).

In an embodiment, the mixture comprising ethanol and at least one C₃ alcohol is treated by a separation system prior to the step of contacting the mixture with a dehydration catalyst. In an embodiment, the separation system comprises at least first and second separation units. In an embodiment, the separation system comprises 2 or more separation units, such as 2 to 10 separation units, 2 to 9 separation units, 2 to 8 separation units, 2 to 7 separation units, 2 to 6 separation units, 2 to 5 separation units, 2 to 4 separation units, 3 separation units, 4 separation units, 5 separation units, 6 separation units, 7 separation units, 8 separation units, 9 separation units, or 10 separation units. In an embodiment, the first separation unit is an evaporator, a distillation column, a set of distillation columns, a combination of a centrifuge and a distillation column, or a combination of a centrifuge and a set of distillation columns. In an embodiment, the second separation unit is a rectifier column, a distillation column, or a set of distillation columns.

In an embodiment, the mixture comprising ethanol and at least one C₃ alcohol is treated by a distillation system prior to the step of contacting the mixture with a dehydration catalyst. In an embodiment, the distillation system comprises 2 or more distillation columns, such as 2 to 10 distillation columns, 2 to 9 distillation columns, 2 to 8 distillation columns, 2 to 7 distillation columns, 2 to 6 distillation columns, 2 to 5 distillation columns, 2 to 4 distillation columns, 3 distillation columns, 4 distillation columns, 5 distillation columns, 6 distillation columns, 7 distillation columns, 8 distillation columns, 9 distillation columns, or 10 distillation columns.

In an embodiment, the ethanol and C₃ alcohol(s) are isolated from a fermentation broth and a fermentation off-gas, the isolation process comprising flowing a fermentation off-gas coming from one or more fermenters through a product recovery unit wherein the flow of the off-gas is counter to a solvent flowing through the product recovery unit and obtaining a solvent stream comprising the at least one C₃ alcohol and ethanol, and mixing the solvent stream with a fermentation broth from the one or more fermenters. In an embodiment, the solvent is water. In an embodiment, the process further comprises passing the fermentation broth through a first distillation column to generate a bottom stream comprising water, heavy components, and solids, and a side stream comprising ethanol, the at least one C₃ alcohol, and water. In an embodiment, the side stream from the first distillation column is passed through a second distillation column to generate a bottom stream comprising water and a side stream comprising ethanol, the at least one C₃ alcohol, and water. In an embodiment, the side stream from the second distillation column is passed through a third distillation column if acetone is present at a concentration higher than 2% by weight of the total weight of the mixture, wherein the acetone is recovered at the top of the third distillation column and ethanol, the at least one C₃ alcohol, and water are recovered at the bottom of the distillation column.

In an embodiment, water is further removed by a drying/dewatering unit after the distillation system and before contacting the mixture comprising ethanol and at least one C₃ alcohol with a dehydration catalyst. In an embodiment, the drying/dewatering unit comprises a membrane separation system. In an embodiment, the drying/dewatering unit comprises a molecular sieve system. In an embodiment, the drying/dewatering unit comprises a membrane separation system and a molecular sieve system. In an embodiment, the mixture comprising ethanol and at least one C₃ alcohol is treated with an ionic resin system to remove salts after the drying/dewatering unit and before contacting the mixture with a dehydration catalyst. In an embodiment, the ionic resin system comprises a cationic resin, an anionic resin, or a combination thereof.

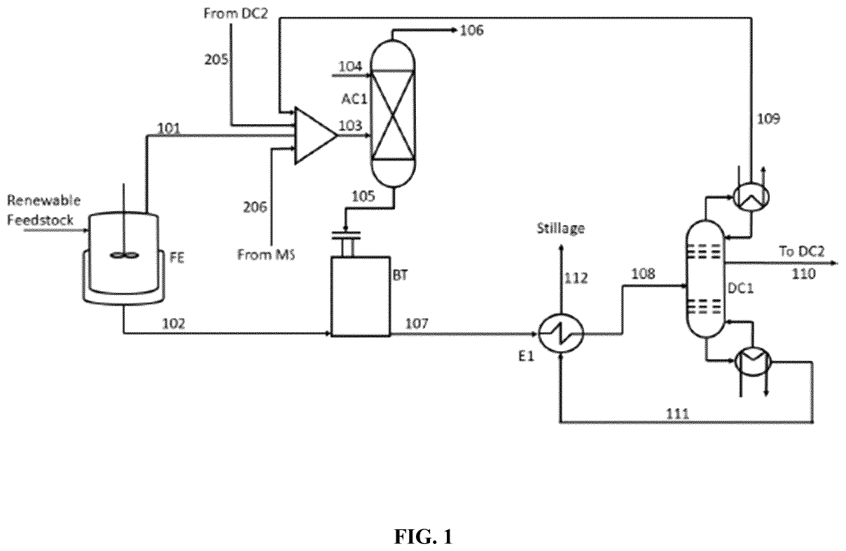

With reference to FIG. 1, a fermentable carbon source (e.g., a renewable feedstock) enters the fermenter (FE) in order to undergo the fermentation step, wherein the genetically modified yeast produces at least one C₃ alcohol along with ethanol. The output of this fermentation process leaves the fermenter (FE) either in the off-gas (vapor phase, 101) or in the broth (liquid phase, 102). Typically, the off-gas (101) contains incondensable gases (mainly CO₂) in a concentration between 92% w/w to 98% w/w, water in a concentration between 0.5% w/w to 3% w/w, and the products in a concentration between 2% w/w to 5% w/w (preferably 2.5% w/w). The fermentation broth (102) typically will contain water, the target C₃ alcohol product(s) in a range of concentration between 5 g/L and 30 g/L, the ethanol as the main product in a concentration between 40 g/L and 140 g/L, and other components present in smaller amounts than the main products, here called contaminants or congeners. The later were already present in the feedstock or were produced by the fermentation process, and may be organic acids, organic alcohols, proteins, saccharides, fats, minerals, and fibers for example.

For all cases wherein the C₃ alcohol is 1-propanol and/or 2-propanol, as shown in FIG. 1, the fermentation off-gas always undergoes a product recovery unit, in which the products present in the off-gas are absorbed by the water added at the top of the equipment (AC1); along with fermentation off-gas, vapors coming from the top of DC1 (109), the top of DC2 (205) and from MS (206) can also undergo this step in AC1, minimizing product loss in vapor phase. An absorption column or preferably a scrubber column can be used in this step, in which the off-gas and vapors from DC1, DC2 and MS combined (103) flow in preferably counter current with a solvent such as water in a proportion of 0.5 to 2 parts of water for one part of off-gas in a mass basis. This process produces a purified gas stream with small quantities of products and a water stream with recovered C₃-products. The solvent used in this is step (104) is preferably, but not limited to, water and must be at a temperature in a range of 5° C. to 45° C. AC1 produces stream 106 at the top.

The stream containing recovered products (105) is mingled with the fermentation broth (102) forming a mixture typically called beer, which will be stored in the beer tank (BT). The beer (107) is sent to the DC1 separation unit. This beer is preheated by heat integration with stillage (E1) and passes through DC1 separation unit. Stream 107 exchanges heat with stream 111, becoming stream 108, with stream 112 going to stillage. The DC1 separation unit is one distillation column capable of treating solids and is usually called beer or mash column. This step removes water, heavy components, and solids at the bottom of the column DC1, with product loss limited to low levels at the so-called stillage. The stillage can have different end uses, according to the feedstock selected and may have a content of 65% w/w to 99% w/w of water, and the rest will be composed of solids present in the feedstock and C₆-sugars that were not consumed during fermentation. The stream coming out from the top of the DC1 step (109) may be composed from 15% w/w to 60% w/w of low boiling points products, ethanol, and from 40% w/w to 85% w/w of carbon dioxide. The stream from the top of the DC1 step (109) will be recycled to AC1 to recover products and remove incondensable gases. The side stream in DC1 (110) contains products and water both at a concentration in a range of 35% to 80% w/w. If a distillation column is used in the DC1 step, it may operate at a range of pressure between 0.1 and 3 bar, the beer must be at temperature between 70° C. and 115° C., and the side stream at temperature between 70° C. and 135° C. Eventual heat integrations with column condensers will depend on the products being separated.

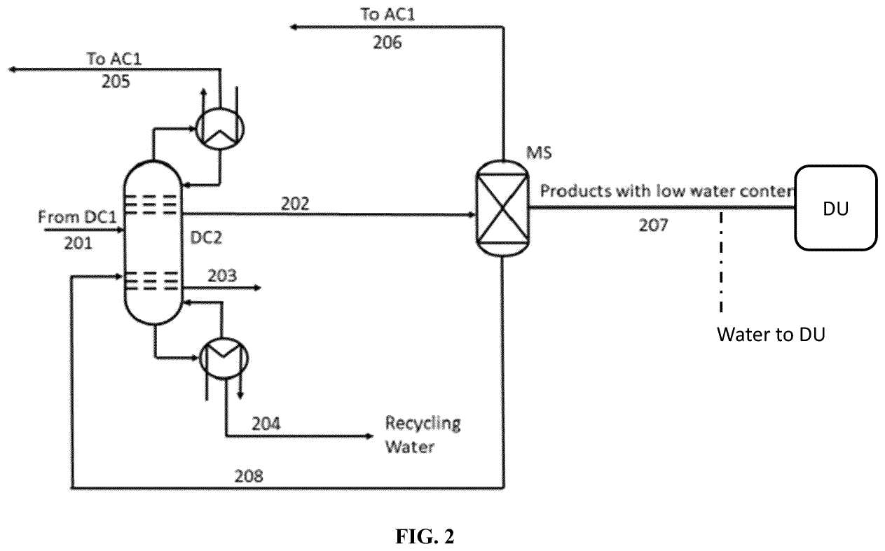

As shown in FIG. 2, this side product stream from DC1 (201) then passes through a second separation unit (DC2), preferably a distillation column, usually called rectifier column. The DC2 bottom stream (204) is mainly composed of water (80% to 100%) at a range of temperature between 65° C. and 135° C. and that water can be recycled to the fermentation process upstream, saving water consumption in the process. The intermediate water content stream (202) of this equipment operates at a range of temperature between 50° C. and 110° C. and it may have from 5% w/w to 10% w/w of water, from 1% w/w to 15% w/w of low-boiling products like acetone, 1-propanol, 2-propanol, congeners, etc., and from 75% w/w to 95% w/w of ethanol. The DC2 distillation column operates at a pressure in a range from 0.1 bar to 3 bar. The vents from the top of DC2 (205) return to the AC1 unit to avoid the loss of desired products. If desired, for specific applications, additional operational units can be added to purify fusel oil stream (203) to be sold as a product. Optionally, the fusel oil stream (203) can undergo a separation phase step, with or without water addition, and the phase rich in water can be recycled to any previous process step; the organic phase—that contains the fusel oil—could be either isolated or could be added to the final ethanol stream without disturbing the ethanol fuel specification. Alternatively, a flash tank could be used to recover acetone, propanol, and ethanol from the fusel oil stream before the separation steps abovementioned. In some process configurations, DC2 could have additional side draws to remove light superior alcohols. In such configurations, additional unit operations may be required to reduce acetone, propanol, and ethanol losses. The intermediate water content stream exiting DC2 (202) is forwarded to a drying/dewatering unit (MS) to have its water content reduced in a range of 2.5% to 0.5% w/w, a key step to operating expense reduction, preservation of product specification, and economic viability. This drying process can be performed by a membrane separation system or any other de-watering industrial equipment or preferably by adsorption using a molecular sieve system. This step is critical for the process when propanol is present because the presence of higher amounts of water will complicate the separation of ethanol from propanol. The water removed in this MS unit may contain any of the products, so this stream (208) is recycled back to the DC2. The stream with low water content leaving the MS (207) is sent to a dehydration unit (DU).

In an embodiment, the separation of ethylene and propylene is achieved through a multi-stage process comprising distillation, filtration, liquid-liquid separation, or a combination thereof.

In an embodiment, ethylene and propylene are polymerized in a reactor using a suitable catalyst system, such as a Ziegler-Natta or metallocene catalyst. The polymerization process is conducted under controlled temperature and pressure conditions to produce polyethylene, polypropylene, and/or a co-polymer of ethylene and propylene.

The resulting polymer is then subjected to purification steps to remove any unreacted monomers and catalyst residues. The purified polymer is characterized by its molecular weight, crystallinity, and mechanical properties, such as tensile strength and elongation at break.

In an embodiment, ethylene and propylene are fed directly into a polymerization reactor without prior separation. The ethylene and propylene can be present in varying proportions. In an embodiment, the ethylene is present at 50% or more of the total alkenes, 60% or more of the total alkenes, 70% or more of the total alkenes, 80% or more of the total alkenes, or 90% or more of the total alkenes, and the propylene is present at 50% or less of the total alkenes, 40% or less of the total alkenes, 30% or less of the total alkenes, 20% or less of the total alkenes, or 10% or less of the total alkenes.

The polymerization is carried out using a suitable catalyst system, such as a Ziegler-Natta or metallocene catalyst, under controlled temperature and pressure conditions. The reaction conditions are optimized to promote the formation of a co-polymer of ethylene and propylene. The resulting co-polymer is then subjected to purification steps to remove any unreacted monomers and catalyst residues. The purified co-polymer is characterized by its molecular weight, crystallinity, and mechanical properties, such as tensile strength and elongation at break.

### EXAMPLES

### General Procedures

All tests reported in the following examples were carried out in a lab-scale isothermal fixed-bed reactor coupled with a gas chromatograph.

### Example 1: Dehydration of Mixture of Ethanol and 2-Propanol

An alumina-based catalyst, as powder, having a surface area of 96 m²/g was tested in the dehydration of a mixture containing ethanol and 2-propanol. Reaction temperature, catalyst mass, and 2-propanol and water concentration were varied. Propylene yield and 2-propanol conversion are shown in Table 1 for this alumina-based catalyst. The results demonstrate that this catalyst was able to convert ≥96% of 2-propanol and yield 100% of propylene between 350° C. and 500° C. CP1, CP2, and CP3 are triplicate at the central point.

TABLE 1
Propylene yield and 2-propanol conversion.

| | | | | | Propylene | 2-Propanol |
|---|---|---|---|---|---|---|
| | Reaction | Catalyst | 2-Propanol Vapor | Water Content | Yield | Conversion |
| No. | Temp. (° C.) | Mass (g) | Pressure (bar) | (vol. %) | (mol %) | (mol %) |
| 1 | 250 | 0.100 | 0.050 | 0 | 21.08 | 24.05 |
| 2 | 470 | 0.100 | 0.050 | 0 | 104.38 | 100 |
| 3 | 250 | 0.250 | 0.050 | 0 | 52.54 | 59.29 |
| 4 | 470 | 0.250 | 0.050 | 0 | 100.39 | 99.95 |
| 5 | 250 | 0.100 | 0.200 | 0 | 2.88 | 5.95 |
| 6 | 470 | 0.100 | 0.200 | 0 | 96.06 | 99.99 |
| 7 | 250 | 0.250 | 0.200 | 0 | 1.75 | 16.81 |
| 8 | 470 | 0.250 | 0.200 | 0 | 95.29 | 99.89 |
| 9 | 250 | 0.100 | 0.050 | 40 | 9.04 | 12.72 |
| 10 | 470 | 0.100 | 0.050 | 40 | 103.78 | 99.99 |
| 11 | 250 | 0.250 | 0.050 | 40 | 14.31 | 21.14 |
| 12 | 470 | 0.250 | 0.050 | 40 | 102.72 | 100.00 |
| 13 | 250 | 0.100 | 0.200 | 40 | 2.01 | 6.39 |
| 14 | 470 | 0.100 | 0.200 | 40 | 95.10 | 99.99 |
| 15 | 250 | 0.250 | 0.200 | 40 | 6.39 | 13.63 |
| 16 | 470 | 0.250 | 0.200 | 40 | 93.25 | 99.95 |
| CP1 | 360 | 0.125 | 0.125 | 20 | 94.31 | 80.37 |
| CP2 | 360 | 0.125 | 0.125 | 20 | 105.42 | 95.60 |
| CP3 | 360 | 0.125 | 0.125 | 20 | 92.27 | 100.00 |

An alumina-based catalyst, as powder, having a surface area of 161 m²/g was tested in the dehydration of a mixture containing ethanol and 2-propanol. Reaction temperature, catalyst mass, and 2-propanol and water concentration were varied. Propylene yield and 2-propanol conversion are shown in Table 2 for this alumina-based catalyst. The results demonstrate that this catalyst was able to convert 100% of 2-propanol and yield 100% of propylene between 250° C. and 300° C. CP1, CP2, and CP3 are triplicate at the central point.

TABLE 2
Propylene yield and 2-propanol conversion.

| | | | | | Propylene | 2-Propanol |
|---|---|---|---|---|---|---|
| | Reaction | Catalyst | 2-Propanol Vapor | Water Content | Yield | Conversion |
| No. | Temp. (° C.) | Mass (g) | Pressure (bar) | (vol. %) | (mol %) | (mol %) |
| 1 | 250 | 0.10 | 0.050 | 0 | 85.44 | 90.24 |
| 2 | 350 | 0.10 | 0.050 | 0 | 102.55 | 100 |
| 3 | 250 | 1.00 | 0.050 | 0 | 102.38 | 100 |
| 4 | 350 | 1.00 | 0.050 | 0 | 100.35 | 100 |
| 5 | 250 | 0.10 | 0.200 | 0 | 26.46 | 37.94 |
| 6 | 350 | 0.10 | 0.200 | 0 | 94.85 | 99.31 |
| 7 | 250 | 1.00 | 0.200 | 0 | 13.42 | 23.79 |
| 8 | 350 | 1.00 | 0.200 | 0 | 92.66 | 100 |
| 9 | 250 | 0.10 | 0.050 | 40 | 45.81 | 48.63 |
| 10 | 350 | 0.10 | 0.050 | 40 | 102.60 | 100 |
| 11 | 250 | 1.00 | 0.050 | 40 | 48.87 | 60.55 |
| 12 | 350 | 1.00 | 0.050 | 40 | 102.07 | 100 |
| 13 | 250 | 0.10 | 0.200 | 40 | 5.01 | 5.87 |
| 14 | 350 | 0.10 | 0.200 | 40 | 88.96 | 90.24 |
| 15 | 250 | 1.00 | 0.200 | 40 | 34.05 | 40.34 |
| 16 | 350 | 1.00 | 0.200 | 40 | 98.34 | 100 |
| CP1 | 300 | 0.55 | 0.125 | 20 | 100.58 | 100 |
| CP2 | 300 | 0.55 | 0.125 | 20 | 100.63 | 100 |
| CP3 | 300 | 0.55 | 0.125 | 20 | 91.97 | 91.37 |

### Example 2: Dehydration of Mixture of Ethanol and 1-Propanol or 2-Propanol

An alumina-based catalyst, as powder, having a surface area of 180 m²/g, pore volume 0.70 cm³/g, was tested in the dehydration of a mixture containing ethanol and 1-propanol or a mixture containing ethanol and 2-propanol. The reaction temperature was 470° C., the catalyst mass was 0.1 g, the pressure was ambient, and weight hourly space velocity (WHSV) was 30 h⁻¹. WHSV is calculated as mass flow rate (g/h)/mass of catalyst (g). Ethanol and propanol conversion are shown in Table 3 for this alumina-based catalyst. The results demonstrate that this catalyst was able to convert up to 100% of mixed alcohols to olefins.

TABLE 3
Ethanol and propanol conversion.

| | | Ethanol | Propanol |
|---|---|---|---|
| Propanol | Weight Ratio of | Conversion | Conversion |
| Component | Ethanol:Propanol | (mol %) | (mol %) |
| 1-Propanol | 1:1 | 99.7 | 99.9 |
| 1-Propanol | 9:1 | 95.7 | 98.4 |
| 2-Propanol | 1:1 | 100 | 100 |
| 2-Propanol | 9:1 | 100 | 100 |

### Example 3: Dehydration of Mixture of Ethanol, 1-Propanol, Water, and Acetone

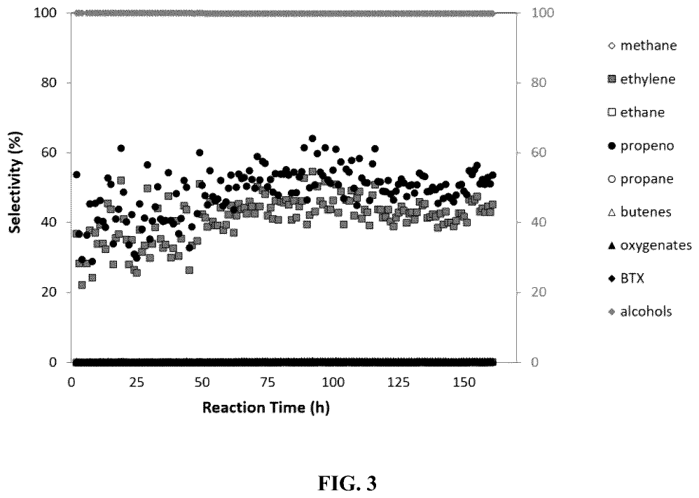

An alumina-based catalyst, as pellets, having a surface area of 180 m²/g, pore volume 0.7 cm³/g was tested in the dehydration of a mixture containing 16.5% wt. ethanol, 16.5% wt. 1-propanol, 67% wt. water, and 50 ppm acetone. The reaction temperature was 470° C., the catalyst mass was 20 g, and the pressure was 6 bar. The results are shown in FIG. 3 and demonstrate that propylene and ethylene formation was not affected by acetone for reaction times up to 160 hours.

### Example 4: Dehydration of Mixture of Ethanol, 2-Propanol, Methanol, and Acetone

An alumina-based catalyst, as powder, having a surface area of 180 m²/g, pore volume 0.7 cm³/g was tested in the dehydration of a mixture containing ethanol and 2-propanol in a weight ratio of 9:1 with up to 2% wt. methanol and up to 2% wt. acetone as contaminants. The reaction temperature was 470° C., the catalyst mass was 0.1 g, the pressure was ambient, and WHSH was 30 h⁻¹. Ethanol and 2-propanol conversion are shown in Table 4 for this alumina-based catalyst. The results demonstrate that this catalyst was able to convert mixed alcohols to olefins.

TABLE 4
Ethanol and 2-propanol conversion.

| | | Ethanol | 2-Propanol |
|---|---|---|---|
| Methanol | Acetone | Conversion | Conversion |
| (wt. %) | (wt. %) | (mol %) | (mol %) |
| 1 | 1 | 100 | 99.23 |
| 1.5 | 0.5 | 100 | 98.71 |
| 2 | — | 100 | 98.75 |
| 0.5 | 1.5 | 100 | 99.05 |
| — | 2 | 99.8 | 97.88 |

### Example 5: Dehydration of Mixture of Ethanol and 1-Propanol or 2-Propanol with Enriched Alumina-Based Catalysts

Two alumina-based catalysts with weak acid sites, as powders, the first having a surface area of 424 m²/g, pore volume 1.05 cm³/g and acidity measured by an NH₃-TPD test of 597 μmol/g (LX065), and the second having a surface area of 401 m²/g, pore volume 1.12 cm³ and acidity measured by an NH₃-TPD test of 710 μmol/g (LX066) were tested in the dehydration of a mixture containing ethanol and 1-propanol or a mixture containing ethanol and 2-propanol. The reaction temperature was 470° C., the catalyst mass was 0.1 g, the pressure was ambient, and weight hourly space velocity (WHSV) was 234 h⁻¹. WHSV is calculated as mass flow rate (g/h)/mass of catalyst (g). Ethanol and propanol conversion are shown in Table 5 for these enriched alumina-based catalysts. The results demonstrate that this catalyst was able to convert around 90% of mixed alcohols to olefins.

TABLE 5
Ethanol and propanol conversion.

| | | | Ethanol | Propanol |
|---|---|---|---|---|
| | Propanol | Weight Ratio of | Conversion | Conversion |
| Sample | Component | Ethanol:Propanol | (mol %) | (mol %) |
| LX065 | 1-Propanol | 1:1 | 86.96 | 94.98 |
| LX065 | 2-Propanol | 1:1 | 87.85 | 94.54 |
| LX066 | 1-Propanol | 1:1 | 91.80 | 94.75 |
| LX066 | 2-Propanol | 1:1 | 92.03 | 96.74 |

In summary, the present disclosure provides methods for the dehydration of alcohols such as ethanol and at least one C₃ alcohol. Compared to existing processes, the disclosed process provides for the lower-cost production, separation, and purification of ethylene and propylene from an ethanol stream. The disclosed process is simpler, more efficient and has reduced capital expenses compared to existing processes since the process described herein has optimized operating expenses, increasing its cost-competitiveness and adoption by industrial ethanol millers.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using consisting of and/or consisting essentially of language. Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A process for production of ethylene and propylene, the process comprising:
(a) contacting a mixture of ethanol and at least one C₃ alcohol with a dehydration catalyst in a dehydration unit at a temperature of 250° C. to 500° C. and at a total pressure of 2 bar to 20 bar to produce ethylene and propylene;
(b) withdrawing from said dehydration unit a stream containing ethylene and propylene produced from step (a); and
(c) separating the ethylene and the propylene from the stream obtained from step (b);
wherein the mixture of ethanol and at least one C₃ alcohol has a water content of 0 to 75% by weight of the total weight of the mixture, wherein the mixture is produced by fermentation using a recombinant yeast in one or more fermenters, and wherein the mixture contains at least 50% ethanol by weight of the total weight of ethanol and C₃ alcohols.

2-5. (canceled)

6. The process of claim 1, wherein the recombinant yeast is an ethanol-producing yeast.

7-8. (canceled)

9. The process of claim 6, wherein the recombinant ethanol-producing yeast comprises: (i) one or more polynucleotides coding for enzymes in a pathway that catalyze a conversion of the fermentable carbon source to succinyl-CoA; (ii) one or more polynucleotides coding for enzymes in a pathway that catalyze a conversion of succinyl-CoA to methylmalonyl-CoA; (iii) one or more polynucleotides coding for enzymes in a pathway that catalyze a conversion of methylmalonyl-CoA to propionyl-CoA; (iv) one or more polynucleotides coding for enzymes in a pathway that catalyze a conversion of propionyl-CoA to propionaldehyde; and (v) one or more polynucleotides coding for enzymes in a pathway that catalyze a conversion of propionaldehyde to 1-propanol.

10. The process of claim 6, wherein the recombinant ethanol-producing yeast comprises: (i) one or more polynucleotides coding for enzymes in a pathway that catalyze a conversion of the fermentable carbon source to 1,2-propanediol; (ii) one or more polynucleotides coding for enzymes in a pathway that catalyze a conversion of 1,2-propanediol to propionaldehyde; and (iii) one or more polynucleotides coding for enzymes in a pathway that catalyze a conversion of propionaldehyde to 1-propanol.

11. The process of claim 6, wherein the recombinant ethanol-producing yeast comprises: (i) one or more polynucleotides coding for enzymes in a pathway that catalyze a conversion of the fermentable carbon source to pyruvate or malonate semialdehyde (MSA); (ii) one or more polynucleotides coding for enzymes in a pathway that catalyze a conversion of pyruvate or MSA to acetyl-CoA; (iii) one or more polynucleotides coding for enzymes in a pathway that catalyze a conversion of acetyl-CoA to acetoacetyl-CoA; (iv) one or more polynucleotides coding for enzymes in a pathway that catalyze a conversion of acetoacetyl-CoA to acetoacetate; and (v) one or more polynucleotides coding for enzymes in a pathway that catalyze a conversion of acetoacetate to acetone; and (vi) one or more polynucleotides coding for enzymes in a pathway that catalyze a conversion of acetone to isopropanol (2-propanol).

12. The process of claim 6, wherein the recombinant ethanol-producing yeast comprises: (i) one or more polynucleotides coding for enzymes in a pathway that catalyze a conversion of the fermentable carbon source to a phosphate intermediate; (ii) one or more polynucleotides coding for enzymes in a pathway that catalyze the conversion of D-xylulose 5-phosphate to D-glyceraldehyde 3-phosphate and acetyl phosphate, or the conversion of D-fructose 6-phosphate to D-erythrose 4-phosphate and acetyl phosphate; (iii) one or more polynucleotides coding for enzymes in a pathway that catalyze the conversion of acetyl phosphate to acetyl-CoA; or one or more polynucleotides coding for enzymes in a pathway that catalyze the conversion of acetyl phosphate to acetate and one or more polynucleotides coding for enzymes in a pathway that catalyze the conversion of acetate to acetyl-CoA; (iv) one or more polynucleotides coding for enzymes in a pathway that catalyze a conversion of acetyl-CoA to acetoacetyl-CoA; (v) one or more polynucleotides coding for enzymes in a pathway that catalyze a conversion of acetoacetyl-CoA to acetoacetate; (vi) one or more polynucleotides coding for enzymes in a pathway that catalyze a conversion of acetoacetate to acetone; and (vii) one or more polynucleotides coding for enzymes in a pathway that catalyze a conversion of acetone to isopropanol (2-propanol).

13. The process of claim 1, wherein the mixture contains at least 70% ethanol by weight of the total weight of ethanol and C₃ alcohols.

14. (canceled)

15. The process of claim 1, wherein the dehydration catalyst is selected from zeolites, oxides, heteropolyacids, or a mixture thereof.

16. The process of claim 15, wherein the dehydration catalyst is a zeolite catalyst is selected from ZSM-5, MOR, or FER.

17. The process of claim 15, wherein the dehydration catalyst is a heteropolyacid catalyst is selected from Ag₃PW₁₂O₄₀, K₂HPW₁₂O₄₀, or Cs₃PMo₁₂O₄₀.

18. The process of claim 15, wherein the dehydration catalyst is an oxide catalyst is selected from gamma alumina, eta alumina, or chi alumina.

19-23. (canceled)

24. The process of claim 1, wherein the mixture is treated by a separation system before contacting the mixture in step (a).

25. The process of claim 24, wherein the separation system comprises at least first and second separation units.

26-27. (canceled)

28. The process of claim 24, further comprising flowing a fermentation off-gas coming from the one or more fermenters through a product recovery unit wherein the flow of the off-gas is counter to a solvent flowing through the product recovery unit and obtaining a solvent stream comprising the at least one C₃ alcohol and ethanol, and mixing the solvent stream with a fermentation broth from the one or more fermenters.

29. The process of claim 28, further comprising passing the fermentation broth through a first distillation column to generate a bottom stream comprising water, heavy components, and solids, and a side stream comprising ethanol, the at least one C₃ alcohol, and water.

30. The process of claim 29, wherein the side stream from the first distillation column is passed through a second distillation column to generate a bottom stream comprising water and a side stream comprising ethanol, the at least one C₃ alcohol, and water.

31. The process of claim 30, wherein the side stream from the second distillation column is passed through a third distillation column if acetone is present at a concentration higher than 2% by weight of the total weight of the mixture, wherein the acetone is recovered at the top of the third distillation column and ethanol, the at least one C₃ alcohol, and water are recovered at the bottom of the distillation column.

32. The process of claim 25, wherein water is further removed by a drying/dewatering unit after the separation system and before contacting the mixture in step (a).

33. (canceled)

34. The process of claim 32, wherein the mixture is treated with an ionic resin system to remove salts after the drying/dewatering unit and before contacting the mixture in step (a).

35-40. (canceled)

41. The process of claim 1, wherein the ethylene and the propylene are polymerized to make polyethylene, polypropylene, and/or a co-polymer of ethylene and propylene.

42-45. (canceled)
